(19) United States
(12) Patent Application Publication
(10) Pub. No.: US 2025/0303027 A1
(43) Pub. Date: Oct. 2, 2025

(54) Methods and Compositions for Tissue Regeneration

(71) Applicant: The General Hospital Corporation, Boston, MA (US)

(72) Inventors: Harald C. Ott, Boston, MA (US); Sydney E. Jeffs, Boston, MA (US); Wafa Altalhi, Boston, MA (US)

(21) Appl. No.: 18/864,294

(22) PCT Filed: May 11, 2023
(86) PCT No.: PCT/US2023/066859

§ 371 Date: Nov. 8, 2024

Related U.S. Application Data
(60) Provisional application No. 63/340,817, filed on May 11, 2022.

(51) Int. Cl. A61L 27/52; A61K 35/42; A61K 35/44; A61L 27/36; A61L 27/54

(52) U.S. Cl. CPC A61L 27/52; A61K 35/42; A61K 35/44; A61L 27/3604; A61L 27/54; A61L 2430/22

(57) ABSTRACT

Disclosed is a method for regenerating and/or repairing lung tissue in a subject. The method comprises administering to a subject in need of lung tissue regeneration and/or repair a composition including (i) a carrier comprising a scaffold-forming material, (ii) cellular material selected from the group consisting of endothelial cells, epithelial cells, mesenchymal stem cells, and mixtures thereof, and (iii) pneumocytes. In one embodiment of the method, the administering is intravenously or intratracheally. The administering can be via airways to the lung. The scaffold-forming material can comprise (i) a first biopolymer having a first reactive group; (ii) a second biopolymer having a second reactive group, wherein the first reactive group and the second reactive group react via click chemistry to crosslink the first biopolymer and the second biopolymer to form a hydrogel; (iii) a porogen; and a (iv) porogen-degrading agent.

### CROSS-REFERENCE TO RELATED APPLICATIONS

This application is based on, claims benefit of, and claims priority to U.S. Application No. 63/340,817 filed on May 11, 2022, which is hereby incorporated by reference herein in its entirety for all purposes.

### STATEMENT REGARDING FEDERALLY SPONSORED RESEARCH

Not Applicable.

### BACKGROUND OF THE INVENTION

### 1. Field of the Invention

The present disclosure relates generally to methods and compositions for tissue regeneration, and more particularly to methods and compositions for lung tissue regeneration. The present disclosure also relates to hydrogel-microsphere scaffolds for generating specific tissue structures. More specifically, the disclosure also relates to click chemistry-driven hydrogel formation with degradable microspheres as scaffolds for generating alveolus-like structures.

### 2. Background and Description of the Related Art

Emphysema, a leading cause of death worldwide, is a risk factor for cardiovascular morbidity. It is characterized by the breakdown of elastin, which permanently enlarges distal airspaces causing destruction of the fragile tissue in the air sacs. This results in the loss of distal tissue alveolar cells and capillaries, thereby leading to disrupted gas exchange and decreased elastic recoil of the lung while increasing lung compliance. Ultimately, air is trapped with increased physiologic dead space. Surgeries can reduce lung volume, but no other therapeutic option can improve lung function and regenerate lost tissue. While there are treatment options to manage the disease, there does not exist a cure. Existing procedures to treat the disease such as lung transplantation and lung volume reduction surgery present high risk.

Therefore, there is a need for minimally invasive and more effective treatment options for emphysema.

### SUMMARY OF THE INVENTION

The present invention meets the foregoing needs by providing methods and compositions for tissue regeneration, and more particularly to methods and compositions for lung tissue regeneration.

In one aspect, the disclosure provides a method for regenerating and/or repairing lung tissue in a subject. The method comprises administering to a subject in need of lung tissue regeneration and/or repair a composition including (i) a carrier comprising a scaffold-forming material, (ii) cellular material selected from the group consisting of endothelial cells, epithelial cells, mesenchymal stem cells, and mixtures thereof, and (iii) pneumocytes. In one embodiment, the cellular material comprises endothelial cells. In one embodiment of the method, the administering is intravenously or intratracheally. The administering can be via airways to the lung.

In yet another aspect, the disclosure provides an injectable composition for forming a scaffold, in which the composition comprises a carrier comprising a scaffold-forming material; cellular material selected from the group consisting of endothelial cells, epithelial cells, mesenchymal stem cells, and mixtures thereof; and pneumocytes. The composition can be used in the treatment of a lung condition. The composition can be used in the treatment of emphysema. In one embodiment, the cellular material comprises endothelial cells. In one embodiment, the endothelial cells comprise induced pluripotent stem cell-derived endothelial cells. In one embodiment, the pneumocytes comprise induced pluripotent stem cell-derived pneumocytes. In one embodiment, the scaffold-forming material comprises a hydrogel. The present disclosure also provides a kit for use in in producing a tissue scaffold. The kit comprises a container; and an amount of the composition in the container. The container can be a medical syringe.

In yet another aspect, the disclosure provides an injectable composition for forming a scaffold. The composition comprises: (i) a biopolymer having a first reactive group and a second reactive group, wherein the first reactive group and the second reactive group react via click chemistry to crosslink the biopolymer to form a hydrogel; (ii) polysaccharide microspheres; and a (iii) polysaccharide-lyase. The present disclosure also provides a kit for use in in producing a tissue scaffold. The kit comprises a container; and an amount of the composition in the container. The container can be a medical syringe.

In yet another aspect, the disclosure provides an injectable composition for forming a scaffold. The composition comprises: (i) a first biopolymer having a first reactive group; (ii) a second biopolymer having a second reactive group, wherein the first reactive group and the second reactive group react via click chemistry to crosslink the first biopolymer and the second biopolymer to form a hydrogel; (iii) polysaccharide microspheres; and a (iv) polysaccharide-lyase. The present disclosure also provides a kit for use in in producing a tissue scaffold. The kit comprises a container; and an amount of the composition in the container. The container can be a medical syringe.

In yet another aspect, the disclosure provides a therapeutic method of providing a scaffold in a tissue environment in the body of a subject. The method comprises injecting any composition of the present disclosure into the tissue environment; and allowing the composition to solidify and degrade. The scaffold can comprise a biodegradable, biocompatible alveolus-like structure. The tissue can comprise lung tissue. The injecting can be intra-tracheally into the lung(s) of the subject. The method can be a therapeutic treatment for emphysema.

These and other features, aspects and advantages of various embodiments of the present disclosure will become better understood with regard to the following description, appended claims, and accompanying Figures.

### BRIEF DESCRIPTION OF THE DRAWINGS

FIG. 1 shows the experimental mode I validation and induced pluripotent stem cells (iPSCs) differentiation. Panel A, Axial computed tomography scan and volume rendering of total lung (−200 to −1200 Hu) and hypodense area (−800 to −1000 Hu) volumes 21 days postelastase injections and hematoxylin and eosin staining of distal lung tissue; scale bar 100 μm. Panel B, Total lung volume. Panel C, Emphysema percentage of total lung volume analysis; n=6 per condition; data presented as mean±SE; *P=0.003 and *P=0001, respectively, by Student t test. Panel D, Mean line intercept analysis; n=5; data presented as mean±SE; *P=0.050 by Student t test. Panel E, Bronchioalveolar lavage (BAL) of healthy control and elastase treated lungs at day 21; Giemsa stain of the BAL; and average white blood cell count per sample, n=3; data presented as mean±SE; *P=0.048 by Student t test. Panel F, Cell treatment regimen. Panel G, Endothelial cell differentiation scheme, day-4 imaging of endothelial cell spheres in bright field (scale bar 100 μm); the 3-dimensional construct of immunofluorescent staining of vascular endothelial-cadherin and 4′,6-diamidino-2-phenylindole (scale bar 20 μm), and percentage of cluster of differentiation (CD) 144, CD31, and CD34 positive cells at day 4 of differentiation and CD31 at day 14 postdifferentiation, n=3; data presented as mean±SE. Panel H, Pneumocyte differentiation scheme and imaging of 3-dimensional pneumocyte spheres; note ubiquitously expressed green fluorescent protein under the pneumocytes progenitor marker (NKX2.1 gene) promoter (NKX2.1-GFP) and ubiquitously expressed TdTomato protein under the surfactant (SPC gene) promoter (SPSc-TdTomato) reporters (scale bar 200 μm) and staining of GFP expression in spheres that were paraffin-fixed (scale bar 10 μm). Ctrl, Control.

FIG. 2 shows transplanted cells incorporated into host lung structures formed new perfused vasculature and alveoli and acquired functional phenotype. Panel A, Expression of human leukocyte antigen 1 (h-HLA 1) in healthy control lungs (scale bar 20 μm). Panel B, Human cell incorporation across multiple human alveoli in rat lungs (green arrows are h-HLA I-positive cells; red arrows are red blood cells); scale bar 50 μm. Panel C, Human cells incorporation in single alveolus stained with h-HLA1 and

Ulex europaeus agglutinin-I labeled cells (UEA-I); yellow arrows are for human endothelial cells; green arrows are for nonendothelial human cells; scale bar 20 μm. Panel D, In the percentage of h-HLA 1-positive cells in total lung cells; n=5; data presented as mean±SE. Panel E and Panel F, Cross-section of lung showing the distribution of ubiquitously expressed green fluorescent protein under the pneumocytes progenitor marker (NKX2.1 gene) promoter (NKX2.1-GFP) positive; arrow points at positive cells (scale bars 75 and 10 μm, respectively). Panel G and Panel H, Cross-section of lung showing the distribution of h-CD3 I positive cells; arrows point at positive cells (scale bars 75 and 10 μm, respectively). Panel I, Dextran-perfused vasculature; arrows point at h-CD3 I-positive cells (scale bar 10 μm). Panel J, Transmission electron microscopy of h-HLA I or h-CD3 I stained cells detected by 3,3′-diaminobenzidine (DAB) staining of lung sect ions (h-HLA1-DAB) or (h-CD31-DAB) positive cells; scale bar 2 μm. Highlighted cells, blue (endothelial cells); green (pneumocytes) are positive for our markers. Panel K and Panel L, High-power image of NKX2. I-GFP-positive and h-CD3 I-positive cells per an alveolus, arrows point at positive cells. Panel M, Percentage of GFP-positive and h-CD31-positive cells per an alveolus; each dot represents a human cell containing alveolus. Panel N, Frequency of alveoli with >70%, 30%, and <30% of h-HLA I-positive cells human-cell contribution in the structure of randomly selected alveoli. DAPI, 4′,6-Diamidino-2-phenylindole; h-CD31, human-specific anti-CD31; EC, endothelial cells.

FIG. 3 shows cell treatments slow emphysema progression, improve vascular density, provide a high pool of proliferative cells and improve alveolar enlargement. Panel A, Computed tomography scan images of an axial view of lungs and volume rendering of total lung volume (−200 to −1200 Hu), vascular density (−300 to −650 Hu), and emphysema hypodense area (−800 to −1000 Hu). Panel B, Lung volume changes over time between days 21 and 49 postelastase injections; n=6; data are mean±SE; 1-way analysis of variance (ANOVA) P=0.006 followed by Tukey post-hoc: cell-treated versus healthy control (ctrl) (P=0.015); emphysema ctrl versus healthy ctrl (P=0.009); and vehicle versus healthy ctrl (P=0.014). Panel C, Hypodense area volume percentage change to normalized to baseline (day 21); n=5; mean±SE; 1-way ANOVA P=0.0007 followed by Tukey post-hoc: *P=0.0002 and &P=0.003. Panel D, Vascular volume percentage of total lung volume (vascular density) across conditions on day 49; n=3; mean±SE; 1-way ANOVA P=0.000009, followed by Tukey posthoc: *P=0.0001 and &P=0.0001, #P=0.0008, **P=0.013, and ***P=0.020. Panel E, 5-ethynyl-2′-deoxyuridine (EdU) incorporation in human and rat cells; n=3; mean±SE; *P=0.015 with student t test. Panel F, EdU incorporation in rat cells of cell-treated lungs versus emphysema-control lungs; n=3, mean±SE; *P=0.023. Panel G, Alveolar area in μm² individual point represents an alveolus; 30 points per condition, n=3; mean±SE; 1-way ANOVA P=0.009 followed by Tukey post-hoc: *P=0.17; **P=0.0212; and ***P=0.046 versus healthy control. h-HLA1, Human leukocyte antigen 1.

FIG. 4 shows cell treatment improves ventilation mechanics. Panel A, Isolated lung recruited with air; highlighted are the area of signs of air trapping. Panel B, Isolated lung dynamic compliance across conditions; n=5; significance was determined by 1-way analysis of variance (ANOVA) P=5.07e-14, followed by Tukey post-hoc: *P=0.79; **P=4.3e-07; and ***P=1.0 1e-04. Panel C, Residual volume measurements across conditions; n=5; mean±SE; significance was determined by 1-way ANOVA P=0.042; *P=0.89; **P=0.011; and ***P=0.02 1. Panel D, Confocal images of human leukocyte antigen 1-positive cells and elastin in cell treated lungs; scale bar 100 μm. Panel E, Oxygen and carbon dioxide diffusion analysis across conditions after 100% oxygen compared with atmospheric air exposure; at 100% oxygen exposure, P_{CO2} 1-way ANOVA P=0.6, and P_{O2} 1-way ANOVA P=0.28; at atmospheric air, P_{CO2} 1-way ANOVA P=0.33, and P_{O2} 1-way ANOVA P=0.02; *P=0.75; **P=0.02; and ***P=0.05. ctr/, Control; h-HLA1, human leukocyte antigen 1.

FIG. 5 shows the preparation of a hydrogel and cells mixture and injection into a rat.

FIG. 6 shows a summary of the Example 1 study of intratracheal transplantation of induced pluripotent stem cells-derived endothelial cells and pneumocytes in an elastase induced emphysema model mediated lung repair by decreasing emphysema progression and improving the vascular density of treated lungs.

FIG. 7 shows a scheme for the gelation of a hydrogel according to the disclosure. The scheme shows a representation of the click reaction between gelatin fibers modified with norbornene (GeIN) and gelatin fibers modified with tetrazine (GelT). The click reaction results in the loss of N₂ and the formation of a cross-linked hydrogel.

FIG. 8 shows a scheme for generating a composite scaffold that includes the crosslinked click hydrogel, alginate microspheres, and alginate lyase. Including the alginate lyase results in the controlled degradation of the alginate microspheres. The porous scaffold formed by the crosslinked click hydrogel around the microspheres remains once the alginate microspheres are degraded. In the last step, alveolar spheres are formed by encapsulated or engrafted cells growing on the scaffold that has maintained the shape of the microspheres.

FIG. 9 shows a scheme representing endobronchial catheter delivery by injection of the composite scaffold mixture into emphysematic alveoli.

### DETAILED DESCRIPTION OF THE INVENTION

Before the present invention is described in further detail, it is to be understood that the invention is not limited to the particular embodiments described. It is also to be understood that the terminology used herein is for the purpose of describing particular embodiments only and is not intended to be limiting. The scope of the present invention will be limited only by the claims. As used herein, the singular forms “a”, “an”, and “the” include plural embodiments unless the context clearly dictates otherwise. A “subject” is a mammal, preferably a human. By “injectable”, we mean a composition may be delivered to a site by way of a medical syringe. By “crosslink”, we mean the functional groups of a polymer may crosslink with the functional groups of the same polymer or another polymer.

It will be appreciated by those skilled in the art that while the disclosed subject matter is described herein in connection with particular embodiments and examples, the invention is not necessarily so limited, and that numerous other embodiments, examples, uses, modifications and departures from the embodiments, examples and uses are intended to be encompassed by the claims attached hereto.

It should be apparent to those skilled in the art that many additional modifications beside those already described are possible without departing from the inventive concepts. In interpreting this disclosure, all terms should be interpreted in the broadest possible manner consistent with the context. Variations of the term “comprising”, “including”, or “having” should be interpreted as referring to elements, components, or steps in a non-exclusive manner, so the referenced elements, components, or steps may be combined with other elements, components, or steps that are not expressly referenced. Embodiments referenced as “comprising”, “including”, or “having” certain elements are also contemplated as “consisting essentially of” and “consisting of” those elements, unless the context clearly dictates otherwise.

The present invention provides a method for regenerating and/or repairing lung tissue in a subject. The method comprises administering to a subject in need of lung tissue regeneration and/or repair a composition including (i) a carrier comprising a scaffold-forming material, (ii) cellular material selected from the group consisting of endothelial cells, epithelial cells, mesenchymal stem cells, and mixtures thereof; and (iii) pneumocytes. In one embodiment, the endothelial cells comprise induced pluripotent stem cell-derived endothelial cells. The endothelial cells can be cultured prior to administration of the composition. In one embodiment, the epithelial cells comprise induced pluripotent stem cell-derived epithelial cells. In one embodiment, the mesenchymal stem cells comprise induced pluripotent stem cell-derived mesenchymal stem cells. The pneumocytes can comprise induced pluripotent stem cell-derived pneumocytes. The pneumocytes can comprise surfactant protein-C-positive pneumocytes. In one embodiment of the method, the administering is intravenously or intratracheally. The administering can be via airways to the lung.

In one embodiment of the method, the scaffold-forming material comprises a hydrogel. The scaffold-forming material can comprise (i) a biopolymer having a first reactive group and a second reactive group, wherein the first reactive group and the second reactive group react via click chemistry to crosslink the biopolymer to form a hydrogel; (ii) a porogen; and (iii) a porogen-degrading agent. Click chemistry encompasses chemical reactions used to couple two compounds together which are high yielding, wide in scope, simple to perform, and can be conducted in easily removable or benign solvents. Examples of click chemistry include the nucleophilic ring opening of epoxides and aziridines, non-aldol type carbonyl reactions, including the formation of hydrazones and heterocycles, additions to carbon-carbon multiple bonds, including Michael Additions, and cycloaddition reactions, such as a 1,3-dipolar cycloaddition reaction (i.e., a Huisgen cycloaddition reaction). One non-limiting example is the reaction between tetrazine and norbornene.

In one embodiment of the scaffold-forming material, the biopolymer comprises gelatin, the porogen comprises polysaccharide microspheres, and the porogen-degrading agent comprises a polysaccharide-lyase. In another embodiment of the scaffold-forming material, the polysaccharide microspheres comprise alginate microspheres, and the polysaccharide-lyase comprises alginate-lyase.

The scaffold-forming material can comprise (i) a first biopolymer having a first reactive group; (ii) a second biopolymer having a second reactive group, wherein the first reactive group and the second reactive group react via click chemistry to crosslink the first biopolymer and the second biopolymer to form a hydrogel; (iii) a porogen; and (iv) a porogen-degrading agent. In one embodiment, one or both of the first biopolymer and the second biopolymer comprises gelatin, the porogen comprises polysaccharide microspheres, and the porogen-degrading agent comprises a polysaccharide-lyase. In one embodiment, the polysaccharide microspheres comprise alginate microspheres, and the polysaccharide-lyase comprises alginate-lyase.

In one embodiment of the method, the lung tissue is emphysematous, and following the administration of the composition, emphysema progression is ameliorated and vascular density of lungs is improved. In one embodiment of the method, the lung tissue is emphysematous, and the method ameliorates emphysema structurally by integrating into host lung tissue and forming blood vessels, and functionally by improving emphysema progression and ventilation. In one embodiment of the method, the lung tissue is emphysematous, and following the administration of the composition, transplanted cells engraft in at least 10% of host alveoli and fully integrate to form vascularized alveoli together with host cells.

The present invention also provides an injectable composition for forming a scaffold, in which the composition comprises a carrier comprising a scaffold-forming material; cellular material selected from the group consisting of endothelial cells, epithelial cells, mesenchymal stem cells, and mixtures thereof; and pneumocytes. The composition can be used in the treatment of a lung condition. The composition can be used in the treatment of emphysema. In one embodiment, the endothelial cells comprise induced pluripotent stem cell-derived endothelial cells. In one embodiment, the endothelial cells are cultured prior to administration of the composition. In one embodiment, the pneumocytes comprise induced pluripotent stem cell-derived pneumocytes. In one embodiment, the pneumocytes comprise surfactant protein-C-positive pneumocytes. In one embodiment, the scaffold-forming material comprises a hydrogel.

In one embodiment, the scaffold-forming material comprises a biopolymer having a first reactive group and a second reactive group, wherein the first reactive group and the second reactive group react via click chemistry to crosslink the biopolymer to form a hydrogel; a porogen; and a porogen-degrading agent. In one embodiment, the biopolymer comprises gelatin, the porogen comprises polysaccharide microspheres, and the porogen-degrading agent comprises a polysaccharide-lyase. In one embodiment, the polysaccharide microspheres comprise alginate microspheres, and the polysaccharide-lyase comprises alginate-lyase.

In one embodiment, the scaffold-forming material comprises: a first biopolymer having a first reactive group; a second biopolymer having a second reactive group, wherein the first reactive group and the second reactive group react via click chemistry to crosslink the first biopolymer and the second biopolymer to form a hydrogel; a porogen; and a porogen-degrading agent. In one embodiment, one or both of the first biopolymer and the second biopolymer comprises gelatin, the porogen comprises polysaccharide microspheres, and the porogen-degrading agent comprises a polysaccharide-lyase. In one embodiment, the polysaccharide microspheres comprise alginate microspheres, and the polysaccharide-lyase comprises alginate-lyase.

The present invention also provides a kit for use in in producing a tissue scaffold. The kit comprises a container; and an amount of the composition in the container. The container can be a medical syringe. In one embodiment, the cellular material comprises endothelial cells. In one embodiment, the endothelial cells are present in the amount of the composition in a range of 10 million to 500 million endothelial cells based on a total amount of the composition in the container, and the pneumocytes are present in the amount of the composition in a range of 1 million to 500 million pneumocytes based on the total amount of the composition in the container. In one embodiment, the endothelial cells are present in the amount of the composition in a range of 10 million to 150 million endothelial cells based on a total amount of the composition in the container, and the pneumocytes are present in the amount of the composition in a range of 1 million to 100 million pneumocytes based on the total amount of the composition in the container. In another embodiment, the endothelial cells are present in the amount of the composition in a range of 60 million to 100 million endothelial cells based on a total amount of the composition in the container, and the pneumocytes are present in the amount of the composition in a range of 10 million to 30 million pneumocytes based on the total amount of the composition in the container.

The present invention also provides another injectable composition for forming a scaffold. The composition comprises: a biopolymer having a first reactive group and a second reactive group, wherein the first reactive group and the second reactive group react via click chemistry to crosslink the biopolymer to form a hydrogel; polysaccharide microspheres; and a polysaccharide-lyase. In one embodiment, the biopolymer comprises gelatin. In one embodiment, the biopolymer comprises gelatin fibers. In one embodiment, the cellular material comprises endothelial cells. In one embodiment, the endothelial cells are present in the amount of the composition in a range of 10 million to 500 million endothelial cells based on a total amount of the composition in the container, and the pneumocytes are present in the amount of the composition in a range of 1 million to 500 million pneumocytes based on the total amount of the composition in the container. In one embodiment, the polysaccharide microspheres comprise alginate microspheres, and the polysaccharide-lyase comprises alginate-lyase. In one embodiment, the microspheres have a maximal dimension in a range of 150 μm to 250 μm. The composition may further comprise cellular material selected from the group consisting of endothelial cells, epithelial cells, mesenchymal stem cells, and mixtures thereof; and pneumocytes. In one embodiment, the first reactive group is tetrazine and the second reactive group is norbornene. The composition can be used in the treatment of a lung condition. The composition can be used in the treatment of emphysema. The present invention also provides a kit for use in in producing a tissue scaffold. The kit comprises a container; and an amount of the composition in the container. The container can be a medical syringe. In one embodiment, the cellular material comprises endothelial cells. In one embodiment, the endothelial cells are present in the amount of the composition in a range of 10 million to 500 million endothelial cells based on a total amount of the composition in the container, and the pneumocytes are present in the amount of the composition in a range of 1 million to 500 million pneumocytes based on the total amount of the composition in the container. In one embodiment, the endothelial cells are present in the amount of the composition in a range of 10 million to 150 million endothelial cells based on a total amount of the composition in the container, and the pneumocytes are present in the amount of the composition in a range of 1 million to 100 million pneumocytes based on the total amount of the composition in the container. In another embodiment, the endothelial cells are present in the amount of the composition in a range of 60 million to 100 million endothelial cells based on a total amount of the composition in the container, and the pneumocytes are present in the amount of the composition in a range of 10 million to 30 million pneumocytes based on the total amount of the composition in the container.

The present invention also provides another injectable composition for forming a scaffold. The composition comprises: a first biopolymer having a first reactive group; a second biopolymer having a second reactive group, wherein the first reactive group and the second reactive group react via click chemistry to crosslink the first biopolymer and the second biopolymer to form a hydrogel; polysaccharide microspheres; and a polysaccharide-lyase. In one embodiment, one or both of the first biopolymer and the second biopolymer comprises gelatin. In one embodiment, both the first biopolymer and the second biopolymer comprise gelatin. In one embodiment, one or both of the first biopolymer and the second biopolymer comprises gelatin fibers. In one embodiment, the polysaccharide microspheres comprise alginate microspheres, and the polysaccharide-lyase comprises alginate-lyase. In one embodiment, the microspheres have a maximal dimension in a range of 150 μm to 250 μm. The composition may further comprise cellular material selected from the group consisting of endothelial cells, epithelial cells, mesenchymal stem cells, and mixtures thereof; and pneumocytes. In one embodiment, the first reactive group is tetrazine and the second reactive group is norbornene. The composition can be used in the treatment of a lung condition. The composition can be used in the treatment of emphysema. The present invention also provides a kit for use in in producing a tissue scaffold. The kit comprises a container; and an amount of the composition in the container. The container can be a medical syringe. In one embodiment, the cellular material comprises endothelial cells. In one embodiment, the endothelial cells are present in the amount of the composition in a range of 10 million to 500 million endothelial cells based on a total amount of the composition in the container, and the pneumocytes are present in the amount of the composition in a range of 1 million to 500 million pneumocytes based on the total amount of the composition in the container. In one embodiment, the endothelial cells are present in the amount of the composition in a range of 10 million to 150 million endothelial cells based on a total amount of the composition in the container, and the pneumocytes are present in the amount of the composition in a range of 1 million to 100 million pneumocytes based on the total amount of the composition in the container. In another embodiment, the endothelial cells are present in the amount of the composition in a range of 60 million to 100 million endothelial cells based on a total amount of the composition in the container, and the pneumocytes are present in the amount of the composition in a range of 10 million to 30 million pneumocytes based on the total amount of the composition in the container.

The present invention also provides a therapeutic method of providing a scaffold in a tissue environment in the body of a subject. The method comprises injecting any composition of the present disclosure into the tissue environment; and allowing the composition to solidify and degrade. The scaffold can comprise a biodegradable, biocompatible alveolus-like structure. The tissue can comprise lung tissue. The injecting can be intra-tracheally into the lung(s) of the subject. The method can be a therapeutic treatment for emphysema.

Described herein is a novel injectable hydrogel/microsphere platform to serve as a scaffold for lung tissue regeneration in patients with emphysema by providing a biocompatible, biodegradable alveolus-like structure. In a non-limiting example, the bulk hydrogel platform utilized can comprise biopolymer (e.g., gelatin) fibers modified to include the click moieties as reactive groups, such as tetrazine (T) and norbornene (N) for quick gelation upon mixing and delivery in physiologic environments. Within this bulk hydrogel, polysaccharide (e.g., alginate) microspheres can be incorporated in a size range of 150 to 250 μm. The polysaccharide (e.g., alginate) microspheres can be incorporated within the bulk gel to provide alveolar-like structures to aid in cell organization. For controlled degradation of the polysaccharide (e.g., alginate) microspheres, polysaccharide-lyase (e.g., alginate lyase) can be incorporated within the bulk gel.

To develop a clinically translatable scaffold, two innovations were necessary: (1) to incorporate a bulk gel that is injectable, and (2) to achieve controlled degradation of the microspheres. These objectives are achieved through the use of a click hydrogel which gels upon mixing and by the incorporation of a polysaccharide-specific enzyme for controlled degradation of the incorporated polysaccharide microspheres.

### EXAMPLES

The following Examples are provided to demonstrate and further illustrate certain embodiments and aspects of the present invention and are not to be construed as limiting the scope of the invention. The statements provided in the Examples are presented without being bound by theory.

### Example 1

### Overview of Example 1

Intra-tracheal injection of a cellular mixture composed of endothelial cell and pneumocytes suspended in a hydrogel can improve emphysema and lung mechanics in nude rat models by regenerating the distal lung tissue. iPS-derived endothelial and pneumocytes can be delivered transtracheally as an injectable hydrogel, thereby integrating into the host tissue, and regenerating lost tissue.

Objectives: Pulmonary emphysema is characterized by the destruction of alveolar units and reduced gas exchange capacity. In Example 1, we aimed to deliver induced pluripotent stem cell-derived endothelial cells and pneumocytes to repair and regenerate distal lung tissue in an elastase-induced emphysema model.

Methods: We induced emphysema in athymic rats via intratracheal injection of elastase as previously reported. At 21 and 35 days after elastase treatment, we suspended 80 million induced pluripotent stem cell-derived endothelial cells and 20 million induced pluripotent stem cell-derived pneumocytes in hydrogel and injected the mixture intratracheally. On day 49 after elastase treatment, we performed imaging, functional analysis, and collected lungs for histology.

Results: Using immunofluorescence detection of human-specific human leukocyte antigen 1, human-specific CD31, and anti-green fluorescent protein for the reporter labeled pneumocytes, we found that transplanted cells engrafted in 14.69%±0.95% of the host alveoli and fully integrated to form vascularized alveoli together with host cells. Transmission electron microscopy confirmed the incorporation of the transplanted human cells and the formation of a blood-air barrier. Human endothelial cells formed perfused vasculature. Computed tomography scans revealed improved vascular density and decelerated emphysema progression in cell-treated lungs. Proliferation of both human and rat cell was higher in cell-treated versus nontreated controls. Cell treatment reduced alveolar enlargement, improved dynamic compliance and residual volume, and improved diffusion capacity.

Conclusions: Our findings suggest that human induced pluripotent stem cell-derived distal lung cells can engraft in emphysematous lungs and participate in the formation of functional distal lung units to ameliorate the progression of emphysema.

Lifelong exposure to cigarette smoke and other airborne toxins leads to emphysema, affecting millions of patients as a leading cause of death worldwide. Emphysema causes the progressive destruction of alveolar units, resulting in reduced gas exchange capacity and recurrent bouts of infection and inflammation in the damaged lung tissue [Ref. 1-3].

This tissue loss occurs via the degradation of the elastin fibers in the extracellular matrix through elastase-proteinease imbalance, caused by the release of elastase from activated neutrophils in the lung, [Ref. 4] leading to the damage of epithelial and endothelial cells in the alveoli and to enlargement of alveolar sacs and to regression of capillaries [Ref. 5]. This ultimately results in air trapping and inadequate ventilation and gas exchange [Ref. 6]. Besides surgical volume reduction [Ref. 7] no treatment exists [Ref. 8-9] that can restore lost lung mechanics, lost functional units, or regenerate functional gas exchange tissue.

The discovery of induced pluripotent stem cells (iPSCs) more than a decade ago provided us with a nearly unlimited source of biological building blocks for regenerative medicine approaches. Recent advances in the directed differentiation of iPS cells to pulmonary epithelial cells [Ref. 8, 10-12] and vascular endothelial cells [Ref. 13-14] enable us to generate cells that constitute the alveolar units. Our team had previously achieved the formation of functional lung tissue ex vivo using primary and iPS-derived cells, with the development of functional vasculature [Ref. 13] and alveolar-formation [Ref. 10] as well as the capacity of gas exchange of transplanted engineered lungs [Ref. 15]. We therefore hypothesized that iPS-derived cells could be differentiated toward lung lineages, delivered to the emphysematous distal lung via the airways, and engraft to form new gas exchange tissue.

In Example 1, we successfully delivered human iPS-derived pneumocytes and endothelial cells within a carrier hydrogel transtracheally in a rat emphysema model. The hydrogel was used as a vehicle to improve delivery and to provide a supporting scaffold for the cells in the lung to improve cell retention. We observed engraftment and incorporation of the human cells in the host lungs and attenuation of emphysema progression over the study period of 49 days.

### Materials And Methods

### Animal Handling and Injections

All experiments were conducted on male athymic nude rats from Charles River Laboratories, strain: 316. At the beginning of the study, rat weight was 220 to 250 g and rats were 70 to 83 days old. All animal studies were approved by the Massachusetts General Hospital institutional animal care and use committee and conducted in accordance with the guide for the care and use of laboratory animals. All rats were singly housed and given unrestricted access to water and chow before use. To perform intratracheal injections, rats were anesthetized with 5% isoflurane and then intratracheally intubated with a 16-gauge angiocatheter. After injection, the catheter was removed, and the animals are weaned from sedation and returned to the cage for recovery. To induce emphysema, we injected elastase (Millipore Sigma, catalog No. 32-468-21000U) at a dose of 32 U/100 g in total 0.5 mL intratracheally. The same procedure was applied for cell injection; the total cell-laden hydrogel volume was 1 mL per dose per rat.

### Cell and Gel Mixture Preparation

Eighty million endothelial cells [Ref. 13] and 20 million pneumocytes [Ref. 10] in a total of 700 μL were mixed with 300 μL gel containing: 0.15 mL PureCol (Advanced Biomatrix, No. 5074), 33.75 μL 0.1N sodium hydroxide, 1 μL N-2-hydroxyethylpiperazine-N-2-ethane sulfonic acid, 2.25 μL 7.5% sodium bicarbonate, 0.6 μL Glutamax supplement (Thermo Fisher Scientific), 20.7 μL Ham's F-12, 15 μL 10 × Dulbecco's modified eagle medium, and 75 μL Matrigel (Corning, catalog No. 356231). The primary components of Matrigel are: laminin (˜60%), collagen IV (˜30%), entactin (˜8%) and the heparin sulfate proteoglycan perlecan (˜2-3%). In Matrigel, entactin acts as a crosslinker between the laminin and collagen IV to create a hydrogel. The mixture was prepared fresh before each injection time point and was kept on ice until injection to prevent gel polymerization. FIG. 5 shows an example preparation of a hydrogel and cells mixture and injection into a rat.

### Isolated Lung Dynamic Compliance and Residual Volume

Lungs were harvested following a procedure previously described [Ref. 15]. Briefly, animals were anesthetized, a laparotomy was performed, heparin was administered via direct injection into the inferior vena cava and was allowed to circulate for 5 minutes, and the animals were exsanguinated. A sternotomy was performed, and the lungs were harvested. The heart was removed, and lungs were attached to the tracheal cannula and secured with a tie. Lungs were connected to a small animal ventilator (Harvard Apparatus) and recruited with air. After 1 to 2 minutes, the lungs were ventilated with 2 mL tidal volume and positive and end-expiratory pressure (PEEP) was maintained at 3 cm H₂O; the output pressure readings were recorded in Lab-Chart software. Average peak inspiratory pressure (PIP) was averaged from 10 respiration cycles. The values were used to calculate the dynamic compliance (Cdyn) using the formula: Cdyn=TV/AP, where TV is tidal volume and AP is the difference between PIP and PEEP. For residual volume, we recruited the lungs as described above; then clipped the trachea using an angioclip; then submerged the lungs in saline, then the lungs were removed again, and the clip was removed lo allow the lung to deflate for 10 seconds. Then the lungs were clipped and submerged again in saline. The amount of saline displacement in the 2 processes was weighted and recorded for each lung and then subtracted from lung weight. The first and second readings were used for controls and residual volume, respectively.

### Endothelial Cell Culture and Differentiation

Sendai virus reprogrammed human iPS (hiPS) from foreskin fibroblasts (ATCC, catalog No. ACS-1019; Lot No. 70017757) were cultured in growth factor reduced Matrigel (Corning, catalog No. 356231) coated 100-cm plates and were maintained in mTESR medium (Stemcell Technologies, catalog No. 100-0276) until confluence. The differentiation was performed in 6-well ultra-low adhesion plates (Corning, catalog No. 3471) at a 2% oxygen incubator for the entire period of differentiation. To precondition cells; we seeded 20,000 cells/cm and fed cells with mTESR medium and 50 uM Y-27632 (hydrochloride) (Cayman Chemical, catalog No. NCO 140250) for 24 hours. The next day, cell aggregates were transferred to a conical tube and allowed to precipitate with gravity; the supernatant was removed and replaced with a mesodermal differentiation medium that contained: 50% DMEM/FI2 (Gibco, catalog No. 11330-032), 50% Neurobasal medium (Gibco, catalog No. 21103049), 1× glutamax (Gibco, catalog No. 35050061), Nonessential amino acids (NEAAs; Gibco, catalog No. 11140035), 1× B27 minus vitamin A (Gibco, catalog No. 12587010), 1× N2 supplement (Gibco, catalog No. 17502048), 46 pg/ul 1-thioglycerol (Sigma, catalog No. M1753), and 10 uM CHIR99021 (Cayman Chemical, catalog No. 4423), then moved to a 2% oxygen incubator for another 24 hours. The following day, cell aggregates were transferred to a conical tube and allowed to precipitate with gravity; the supernatant was removed and replaced with endothelial cell differentiation, which contains Stempro-34 SFM (Gibco, catalog No. 10639011), 200 ng/mL Vascular endothelial growth factor 165 (VEGF-165) (Peprotech, catalog No. 100-20) and 2 nM forskolin (Sigma, catalog No. F3917), then incubates for another 48 hours. After that, cell spheres were taken out from a hypoxic environment and collected in a conical tube, then washed with Dulbecco's phosphate-buffered saline (Gibco, catalog No. 14190235) and allowed to precipitate again. The supernatant was removed, and tryp1E (Gibco, catalog No. 12604039) was added to achieve single cell suspension. Endothelial cells were then purified using CD 144 (Miltenyi Biotec, catalog No. 130-097-857) and LS column filtration (Miltenyi Biotec, catalog No. 130-042-40 I), then seeded on 0.1% gelatin-coated flask in endothelial cell medium, Endothelial Cell Growth Medium 2 (Promocell, catalog No. c-22011) and 10% FBS (Hyclone, catalog No. SH3007002). The cells were expanded at a ratio of 1:3 until we reached 80 million cells per 1 planned rat injection.

### Pneumocyte Cell Culture and Differentiation

The BU3-NGST iPS line was obtained from the Stem Cell Bank at Boston University (https://stemcellbank.bu.edu/Catalog/Ttem/Details/509). The differentiation produces NKX2.1+ progenitors and high or dim surfactant protein C expressing cells [Ref. 10] carrying NKX2.1-green fluorescent protein (GFP) and ubiquitously expressed TdTomato protein under the surfactant (surfactant protein C gene) promoter reporters for lung epithelial progenitor marker and an alveolar type 2 cell marker, respectively. The differentiation to pneumocytes was performed following previously published methods with modifications [Ref. 8]. iPS were maintained in the mTESR medium (Stemcell Technologies). A stepwise differentiation procedure was initiated when cells reached 60% to 70% confluence. The basal medium for all differentiation steps was Dulbecco's modified Eagle's medium (DMEM/F21) (Gibco, catalog No. 11330-032), supplemented with B27 (Gibco, catalog No. 17504044). Endodermal differentiation proceeded using the StemDiff kit (Stemcell Technologies, catalog no. 05110) for 4 days, followed by 4 days of 1 mM A830 I (Sigma, catalog No. NC9890026) and 1 mM IWR-1 (Sigma, catalog No. 1-0160) for anteriorized endodermal differentiation. Ventralized endodermal differentiation proceeded by exposing cells to 10 ng/mL FGF-7 (PeproTech, catalog No. 100-19), 10 ng/ml FGF-10 (PeproTech, catalog No. 100-26), and 3 mM CHIR99021 (Tocris Bioscience, catalog No. 4423) for 7 days. After ventralization, fluorescence-activated cells were sorted for purification of NKX2.1-GFP-positive cells. Sorted NKX2.1+ cells were embedded in 100% Matrigel (Corning, catalog No. 356231) drops to form alveolar spheres. The culture medium for the formation, maintenance, and expansion of the alveolar spheres contained: 49% Medium 199 (Gibco, catalog No. 11150-067), 49% DMEM/F12 (Gibco, catalog No. 11330-032), and 2% fetal bovine serum (Hyclone, catalog No. SH3007002) supplemented with 1×B27 (Gibco, catalog No. 17504044), 10 ng/ml FGF-7 (PeproTech, catalog No. 100-19), 10 ng/ml FGF-10 (PeproTech, catalog No. 100-26), 3 uM CHIR99021 (Tocris Bioscience, catalog No. 4423), 0.1 mM IBMX (Sigma, catalog No. 15879), 0.1 mM 8-Bromo-CAMP (Sigma, catalog No. B7880), 50 nM dexamethasone (Sigma, catalog No. D4902), 10 uM Y-27632 (Calbiochem, catalog No. 688000), and 50 ng/ml ascorbic acid (Sigma, catalog No. 72 132). After a 7- to 14-day culture, Matrigel droplets were digested with 100 μL/droplet of Dispase (Corning, catalog No. 354235) for 1 to 2 hours, then add Dulbecco's phosphate buffered saline and transferred the solution with spheres into a 50-ml conical tube and centrifuge at 1000 rpm for 5 minutes. GFP+TdTomato+cells are then sorted for further expansion. Matrigel-based homogeneous liquid precursor with suspended cells was aliquoted into 100 μL drops containing 20,000 cells each to allow every single cell to form an alveolar sphere during the culture period without sphere overcrowding. Cell-laden Matrigel droplets were drawn into a pipette tip, allowed to warm for 90 seconds, then placed on tissue culture plastic in individual wells of a 12-well plate and allowed to gel at 37° C. for 20 minutes. After Matrigel hardening, 1 mL expansion media was added to each well, and the plate was placed in a 37° C., 5% carbon dioxide incubator. The expansion was performed several times to reach 20 million cells per 1 planned rat injection. On the day of cell transplantation, the Matrigel drops were digested with Dispase and spheres were dispersed using the same procedure mentioned above.

### Computed Tomography Scan and Image Analysis

After the 21-day observation period, rats underwent computed tomography (CT) scanning under sedation on a Siemens Inveon small animal imaging system (Siemens). Rats were imaged using an 80-kVp 500-μA radiograph tube with a complementary metal-oxide semiconductor detector with projection over 360° and reconstructed using a modified Feldkamp one beam reconstruction algorithm (Cobra Exxim) into a 512×512×800 matrix with 113-micron isotropic voxels. Each subject was breathing spontaneously when imaging was obtained. Lung volume and regions of interest analyses were performed using Horos software (horosproject.org). Region of interest tap and lung hyperlucency were detected by selecting an upper (−800) and a lower (−1000) threshold of Hounsfield units. Vascular density was detected by selecting an upper (−300) and a lower (−650) threshold of Hounsfield units.

### Blood Gas Analysis

For blood gas analysis, we used a point of care system, an istat GC8+ (Abbott, No. 03P88-25); rats were exposed to 100% oxygen and 2% isoflurane delivered via nose cone for 10 minutes and the first sample collected from the abdominal aorta, and a GC8+ iStat reading was recorded, then gas was switched to room air for 10 minutes followed by a second sample collected from the abdominal aorta and GC8+ iStat reading recording.

### Isolated Lung Dynamic Compliance and Residual Volume

Lungs were harvested as previously described 2010 [Ref. 13]. Briefly, the animals were anesthetized, a laparotomy was performed, heparin was administered via direct injection into the inferior vena cava and was allowed to circulate for 5 minutes, and the animals were exsanguinated. A sternotomy was performed, and the lungs were harvested. The heart was removed, and lungs were attached to the tracheal cannula and secured with a tie. Lungs were connected to a small animal ventilator (Harvard Apparatus) and recruited with air. After 1 to 2 minutes, the lungs were ventilated with 2 mL tidal volume (TV) and positive a end-expiratory pressure (PEEP) was maintained at 3 cmH₂O; the output pressure readings were recorded in Lab-Chart software. Average peak inspiratory pressure (PIP) was averaged from 10 respiration cycles. The values were used to calculate the dynamic compliance (Cdyn) using the formula: Cdyn=TV/ΔP, where ΔP is the difference between PIP and PEEP. For residual volume, we recruited the lungs as described above; then clipped the trachea using an angioclip; then submerged the lungs in saline, then the lungs were removed again, and the clip was removed to allow the lung to deflate for 10 seconds. Then, the lungs were clipped and submerged again in saline. The amount of saline displacement in the 2 processes was weighted and recorded for each lung and then subtracted from lung weight. The first and second readings were used for controls and residual volume, respectively.

### Perfusion Tracer Studies

Before harvesting the lungs, the abdominal aorta and vena cava were cut to exsanguinate the animals. An incision was then made in the right ventricular outflow tract, the left atrial appendage was removed, and the lungs were flushed with cold phosphate buffered saline to remove blood.

Then perfused with 10 mL 200 μg/mL Ulex europaeus agglutinin I (UEA-1) (Vector laboratories, catalog No. B1065-2) or 100 μg/mL dextran-GFP or dextran-biotin. The solution was kept in the lungs for 10 minutes, and then the lungs were fixed with 4% paraformaldehyde overnight at room temperature, then processed as mentioned later.

### Bronchioalveolar Lavage and White Blood Cells Count

Isolated lungs were injected with 7 mL saline through the trachea. The lavage was collected by resting the lung in a horizontal position to allow passive liquid flow out of the lung. Bronchoalveolar lavage was collected and centrifuged at 300×g for 5 minutes. Then, the pellet was fixed with 4% paraformaldehyde (PFA). An amount of 40 μL of the pellet was spread on a slide and stained with Giemsa staining (Sigma Aldrich catalog No. G5637) as per manufacturer instructions. Images were collected with 40× magnification power.

### Immunofluorescent Staining and Confocal Imaging

For immunohistochemistry staining, samples were fixed in 4% paraformaldehyde and cut arbitrarily at 500 μm thick sections. Cleared with Clarity, clearing kit-low lipid (Clear Light Biotechnologies) following manufacturer instructions. Then, sections were blocked with 1% bovine-specific antigen in phosphate buffered saline for 5 hours and stained with anti-human CD31 (DAKO, catalog No.M082301-2), anti-GFP (Invitrogen, No. CAB4211) or anti-VE-cadherin (R&D systems, No. AF938) and antihuman HLA-1 (DAKO, catalog No. M073601-2) for 2 days at 4° C., followed by 1-day washing in phosphate buffered saline then incubated with A594 (Jackson Immunoresearch, catalog No. 705-586-147), A488 (Jackson Immunoresearch, catalog No. 711-546-152) or A647 (Jackson Immunoresearch, catalog No. 715-606-51) AffiniPure F(ab′)₂ Fragment overnight at 4° C. Sections were then washed again for 3 hours 5 times, and the last wash contained 4′,6-diamidino-2-phenylindole at 1:1000 dilution. After 10 minutes, sections were immersed in mounting medium (life canvas technologies, catalog No. Easy Index_100 mL) and enclosed between a slide and coverslip separated with ·a 100 μm spacer. Stained sections were imaged using SP8 (Leica) or LSM 980 (Zeiss) microscopes. ImageJ was used for image analysis (National Institutes of Health).

### Transmission Electron Microscopy

Following fixation with 4% PFA and permeabilization with 0.01% Triton, ˜1 mm sections were stained with anti-human CD31 or anti-human HLA as described above. Tissue sections were doubled stained with horse anti-mouse HRP secondary antibody (Vector Laboratories, catalog No. PI-2000-1) overnight at 4° C., then with DAB substrate Kit (Abeam, catalog No. ab64238). Tissue sections were washed 2 times with pH 7.4 phosphate buffered saline, then re-fixed in 2.5% glutaraldehyde+3% paraformaldehyde with 5% sucrose in 0.1 M sodium cacodylate buffer (pH 7.4) for 1 hour at 4° C. and postfixed in 1% osmium tetroxide in veronal-acetate buffer then dehydrated and embedded in Embed-812 resin (Electron Microscopy Sciences). Sections were cut on a Reichert Ultracut E microtome with a Diatome diamond knife at 50 nm. Transmission electron microscopy imaging was conducted with an accelerating voltage of 100 kV using a Hitachi 7800 Transmission Electron Microscope.

### Proliferation Detection

Detection of proliferation by labeling cells with 5-ethynyl-2′-deoxyuridine (EdU) (Carbosynth, catalog No. NE08701); 1 mL was injected into the neck flap at a dose of 10 mg/mL for 3 days (days 46, 47, and 48 postelastase injections). Lungs were harvested as described by Ott and colleagues [Ref. 13] then fixed in 4% PFA overnight at 4° C., sectioned and cleared. EdU was detected using click chemistry to covalently attach Alexa Fluor 594 azide to EdU alkyne incorporated into DNA during the S phase of the cell cycle (Invitrogen, catalog No. C 10639; Click-iT Plus EdU Cell Proliferation Kit for Imaging, Alexa Fluor594 dye), by incubating sections in Click-iT reaction cocktail for two hours at room temperature.

### Hematoxylin and Eosin and Verhoeff Van Gieson Staining and Mean Line Intercepts and Alveolar Area Calculations

All samples were fixed with 4% PFA in phosphate buffered saline overnight at room temperature. For hematoxylin and eosin staining, sections were deparaffinized, rehydrated, and stained with hematoxylin (Vector Laboratories) and eosin Y (Fisher Scientific) or with Verhoeff Van Gieson stain (Abeam, catalog No. 150667), then mounted with Permount (Fisher Scientific) after dehydration. Slides were imaged with Easyscan Infinity (Motic microscopy). Slides were analyzed with QuPath software. For Morphometric parameters of healthy control and elastase injected rat lungs, we quantified using the mean linear intersect (MLI), a measure which quantifies alveolar unit size by looking at intersection points on a surface trace of known length on a given lung section. The MLI is a described and accepted method to estimate the approximate free space of the distal lung airspace where a vertical line of known length is overlaid on each image, the number of alveolar and ductal walls intersects, and branch points that pass through the overlaid line were manually recorded. The average intercepts were calculated in 10 to 20 fields per rat for n=5 in each condition. To calculate the alveolar size, we used the QuPath area calculation function in an average of 30 alveoli selected in random areas in the lung left lob cross-section.

### Statistical Analysis

Excel 365 (Microsoft Corporation) was used for data management, statistical analysis, and graph making together with (Graphpad Prism 6 [GraphPad Software]). Statistical analysis was performed by Student t tests (2-tail comparisons) or 1-way ANOVA followed by Tukey post hoc. Values in graphs were presented as means ±SE. Figures were made with Adobe illustrator (Adobe Inc), or created with BioRender.com.

### Results

### Emphysema Formation by Elastase Injection

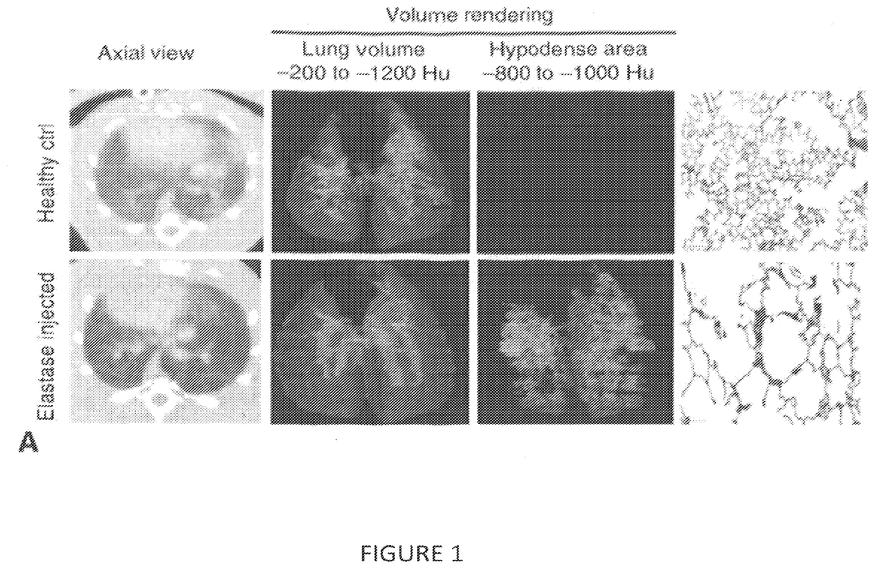

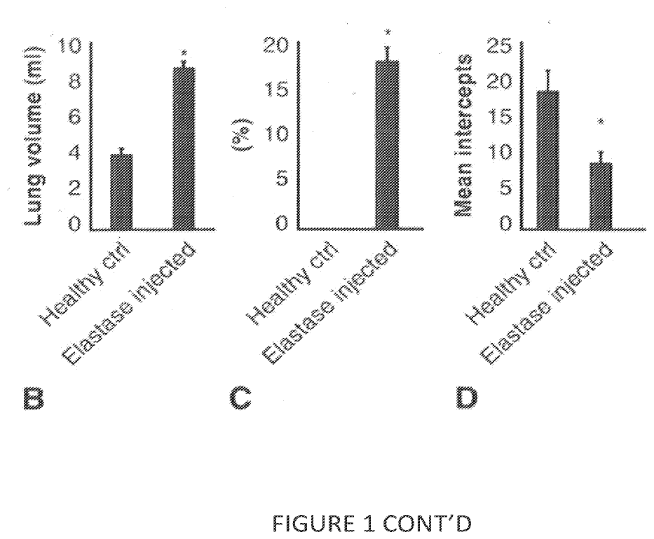

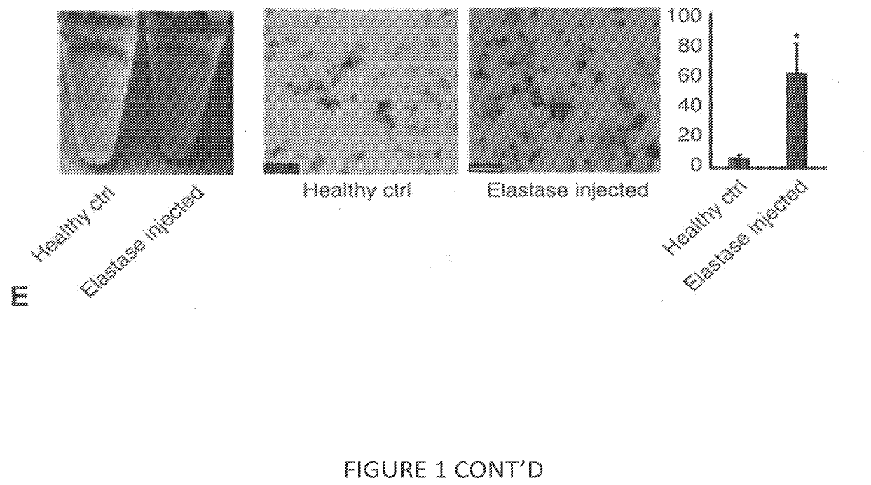

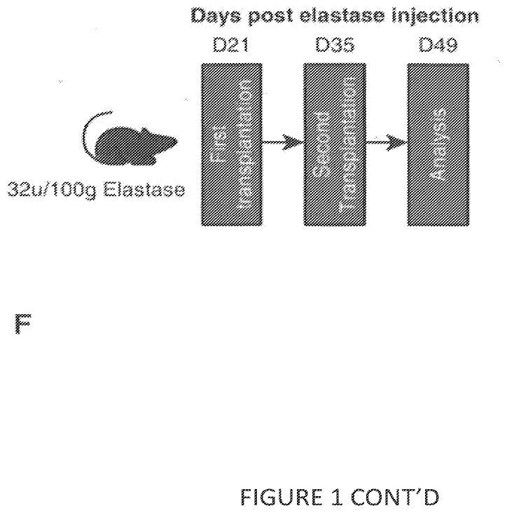

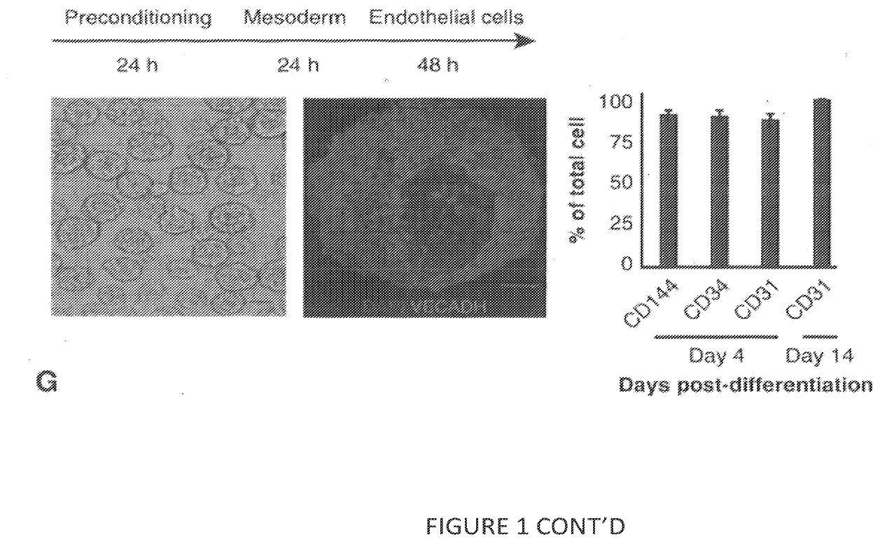

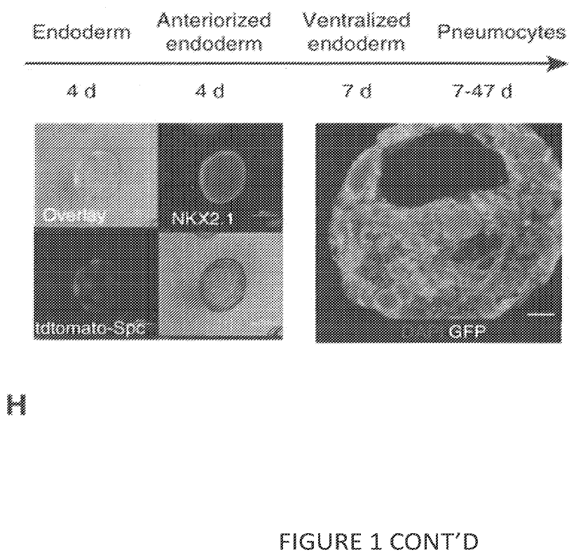

To establish emphysema, we injected 32 U/100 g elastin intratracheally in 220- to 250-g nude rats, as previously reported [Ref. 16]. At 21-days postinjection, we confirmed emphysema formation by axial imaging and histology (FIG. 1, Panel A). In both elastase injected and noninjected healthy controls, we calculated total lung volume (−200 to −1200 Hounsfield units [Hu]) and percentage of the hypodense area (−800 to −1000 Hu) using computed tomography scans, in addition to calculating alveolar density in tissue samples by counting the mean number of septal intercepts of lung tissue against a vertical line (mean linear intercept). We found that the lungs of elastase-injected rats had greater volume compared with the healthy controls (8.56±0.73 mL vs 3.91±0.29 mL, respectively; P=0.003; n=6) (FIG. 1, Panel B), and the hypodense volume of elastase-injected lungs represented 17.16%±1.86% (n=6) of total lung volume (FIG. 1, Panel C). Furthermore, mean±SE linear intercepts in elastase-injected lungs were 8.66±1.6 compared with 18.25±2.9 intercepts in healthy controls (P=0.050; n=3), indicating loss of alveolar architecture, the hallmark of emphysema formation. Elastase-injected lungs further showed evidence of chronic inflammation and loss of barrier function with alveolar bleeding on bronchioalveolar lavage samples, showing higher white blood cells counts in elastase-injected lungs compared with healthy controls (FIG. 1, Panel E) (n=3; 85±32.5 vs 5±3.7 per 40 μL; P=0.048). In the treatment arm, elastase-injected rats received 2 doses of intratracheal cell transplantation: 1 on day 21 and another on day 35. Vehicle-only injected emphysema rats, nontreated emphysema rats, and healthy rats served as controls. At the end of the experiment (on day 49), we performed lung function tests, compliance testing, diffusion testing, and histological analysis (FIG. 1, Panel F).

Directed Differentiation of iPSC-Derived Cells

To generate endothelial cells for injection, we differentiated cells from iPSC in 3-dimensional culture under low oxygen concentration (2%-3% oxygen) [Ref. 14]. On day 4 of differentiation, we observed the formation of endothelial cell spheres with an average of 90% endothelial cells triple positive for cluster of differentiation (CD) 34, CD31, and CD144. (FIG. 1, Panel G). After isolation and expansion, endothelial cell phenotypes were maintained for 5 passages, as determined by flowcytometry detection of CD31, thus suggesting a stable endothelial phenotype. In parallel, we differentiated pneumocytes following a previously published protocol to produce surfactant protein-C-positive pneumocytes [Ref. 12]. We used BU3-NGST iPSCs, [Ref. 11] which carry 2 fluorescent reporters for a lung epithelial progenitor marker and an alveolar type-2 cell marker: ubiquitously expressed green fluorescent protein under the pneumocytes (NKX2.1-GFP) and ubiquitously expressed TdTomato protein under the surfactant (SPC gene) promoter (SPC-TdTomato). After fixation, we used anti-GFP staining to detect GFP (FIG. 1, Panel H), which revealed that 100% of the cells express GFP, enabling us to detect cells in fixed tissue after transplantation. At the end of the differentiation protocol, we produced cells that ubiquitously expressed NKX2.1-GFP, with some cells expressing high SPC-TdTomato, thus suggesting the formation of mature pneumocytes.

### Cell Engraftment in Recipient Lungs

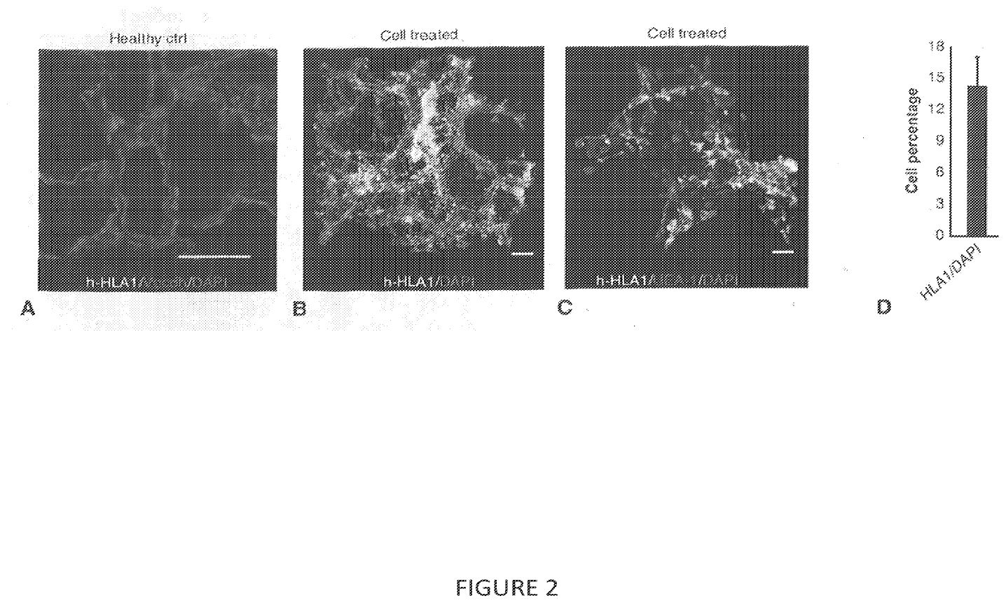

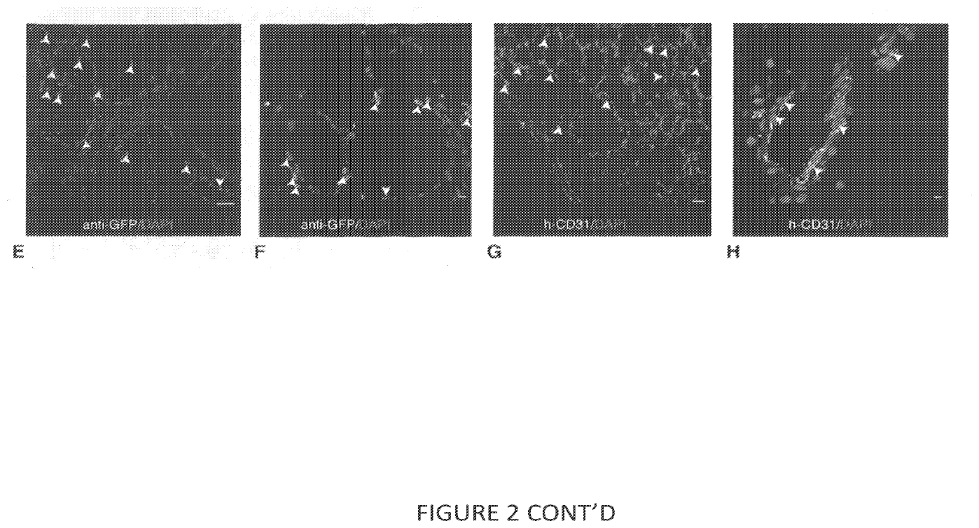

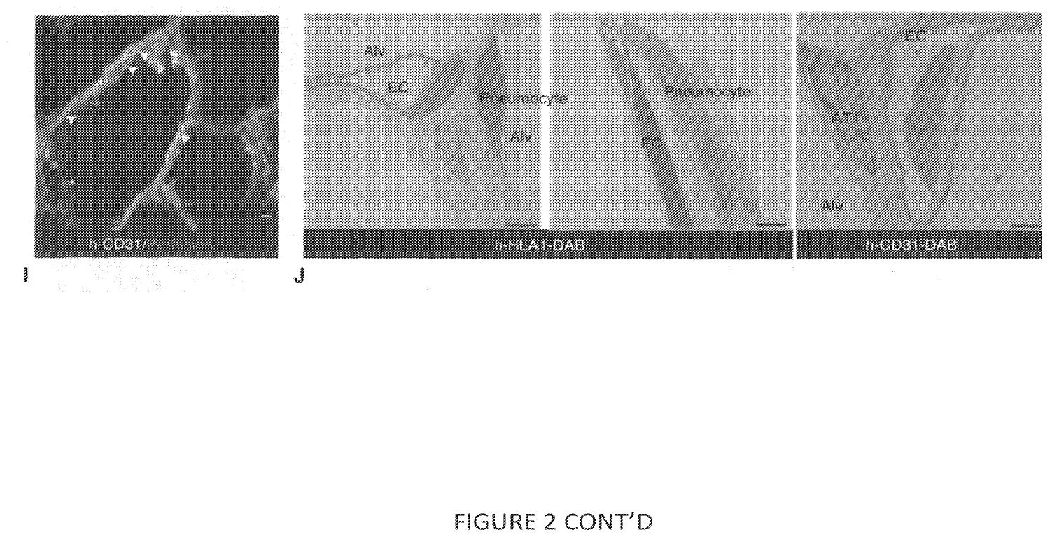

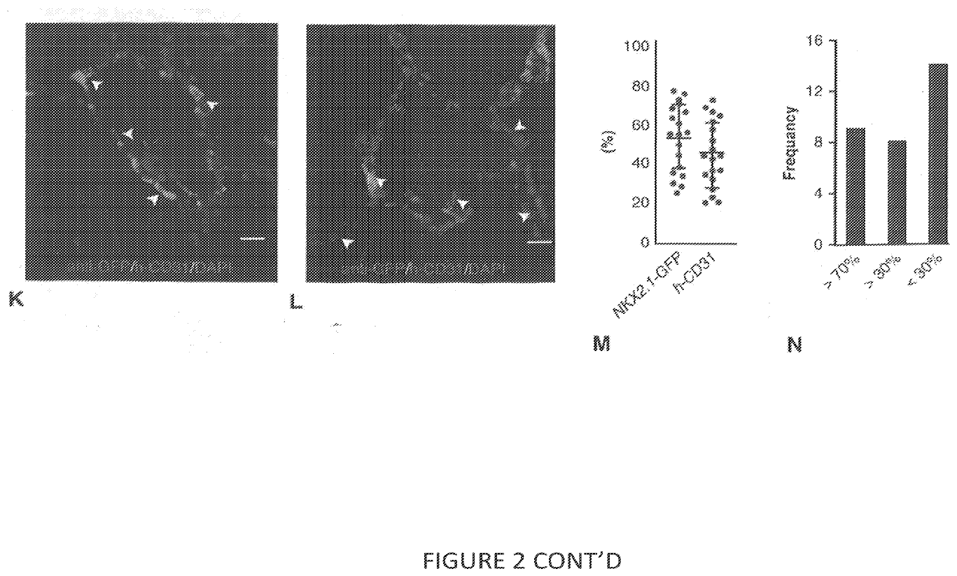

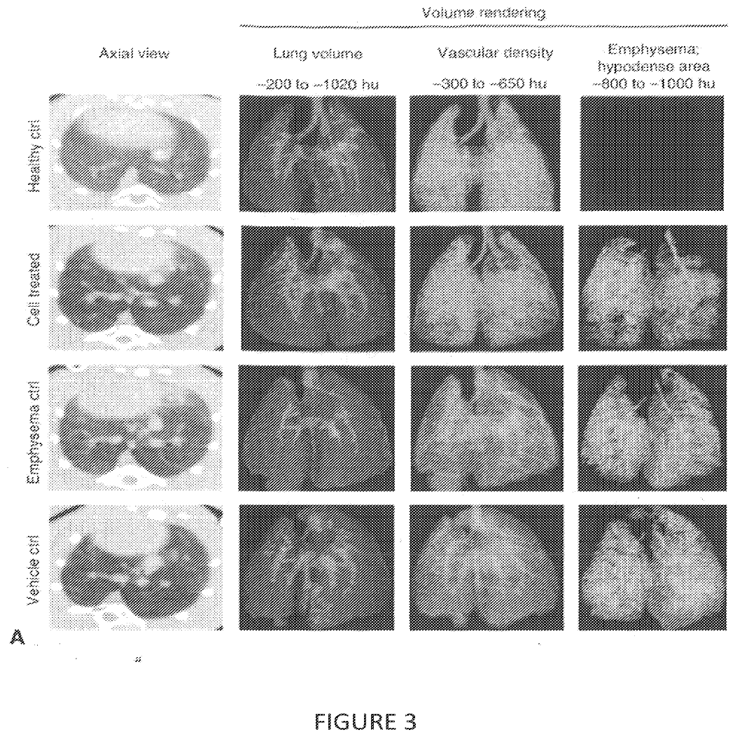

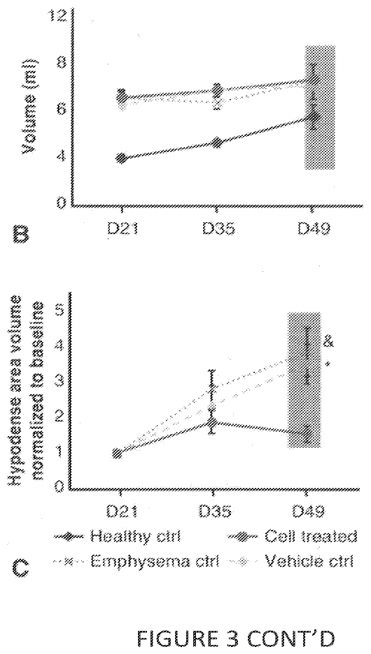

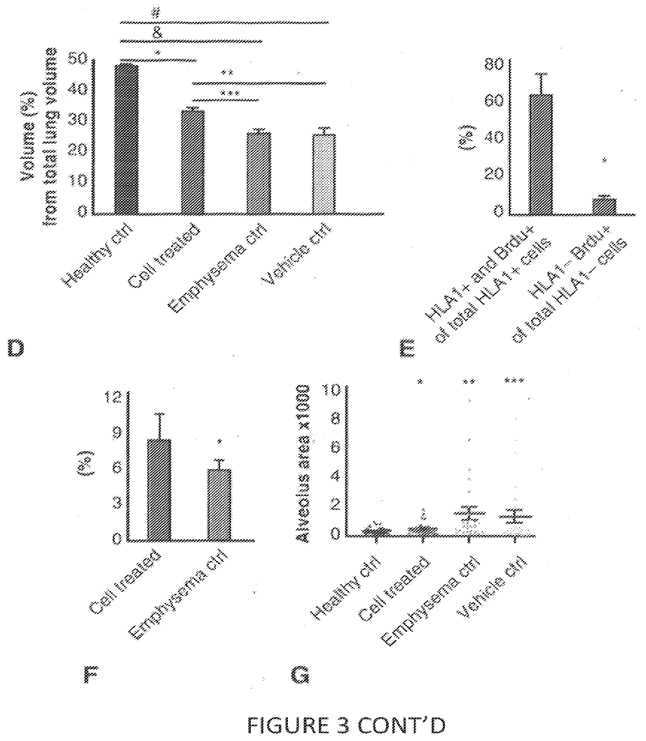

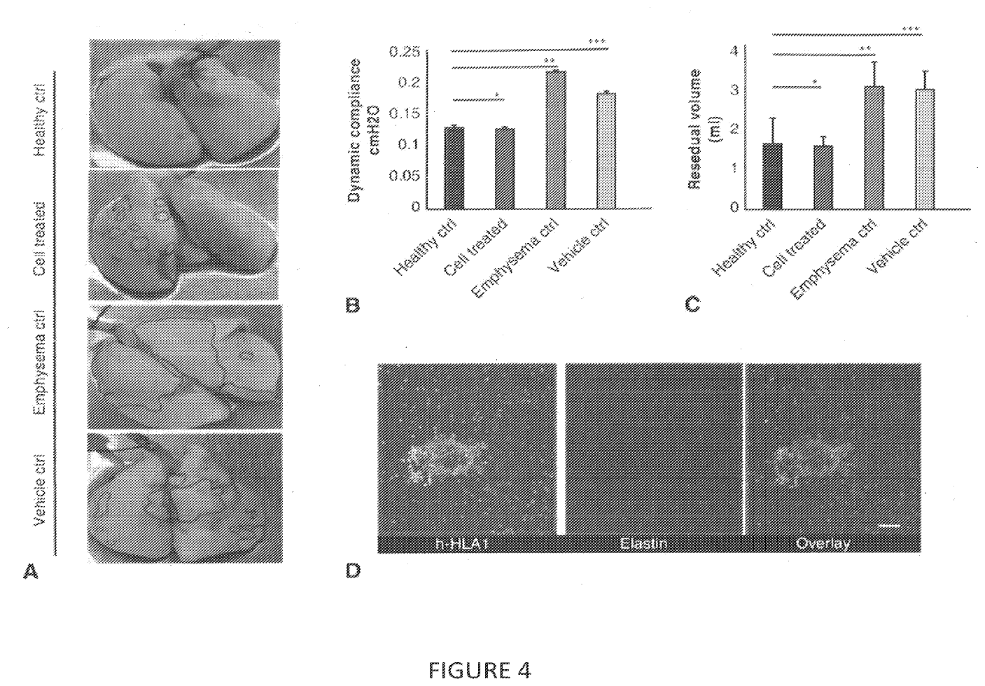

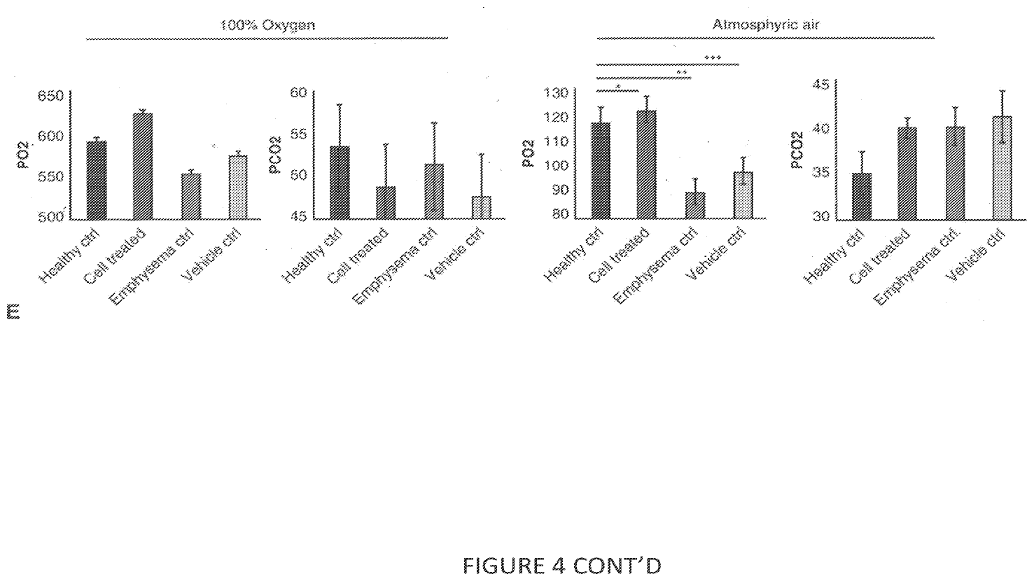

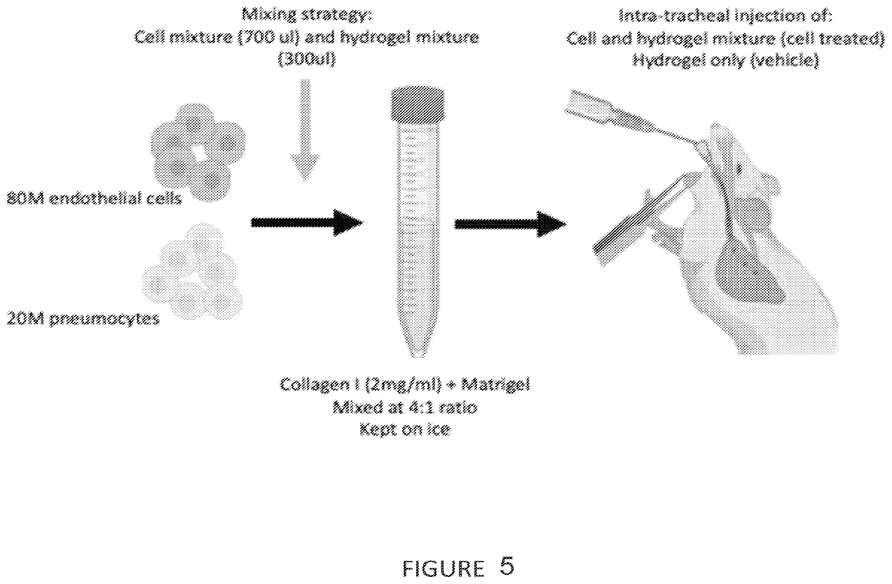

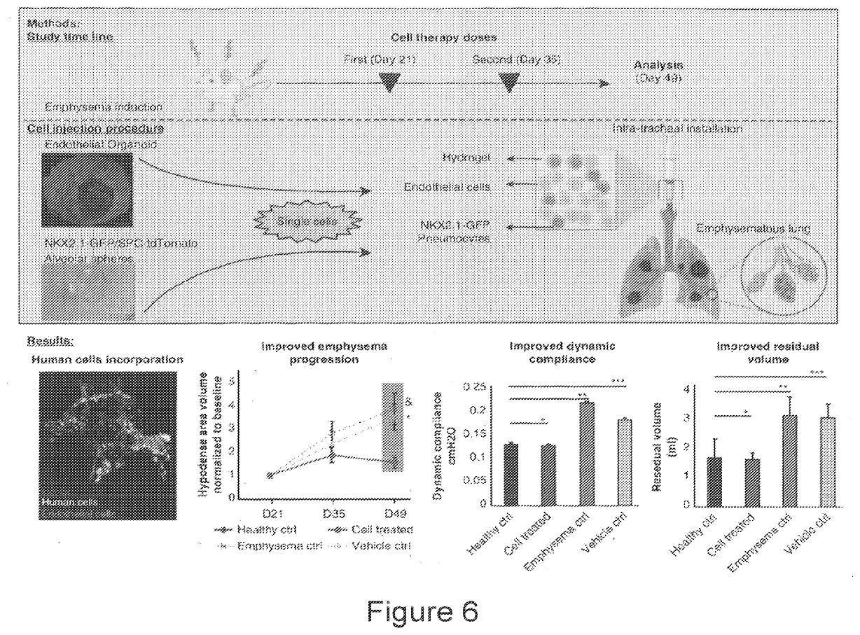

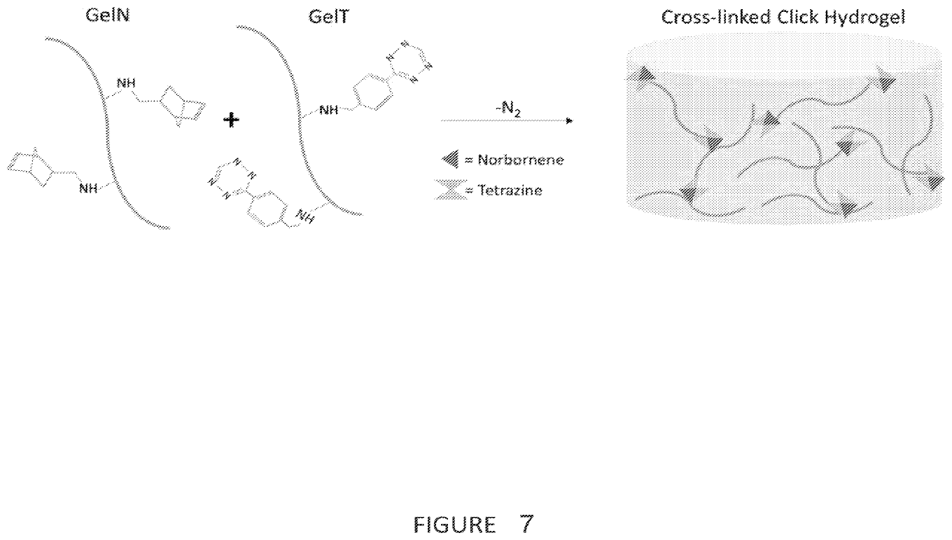

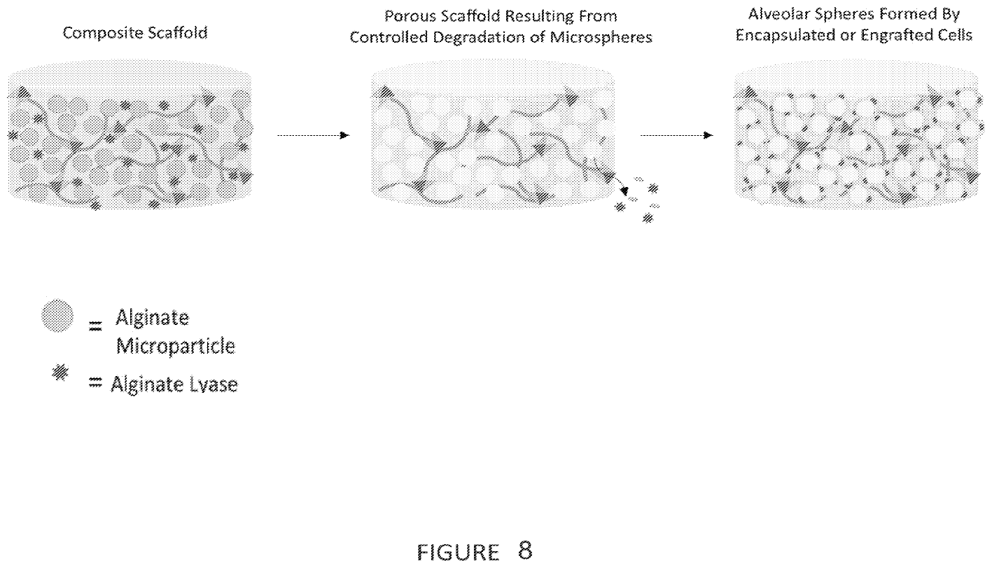

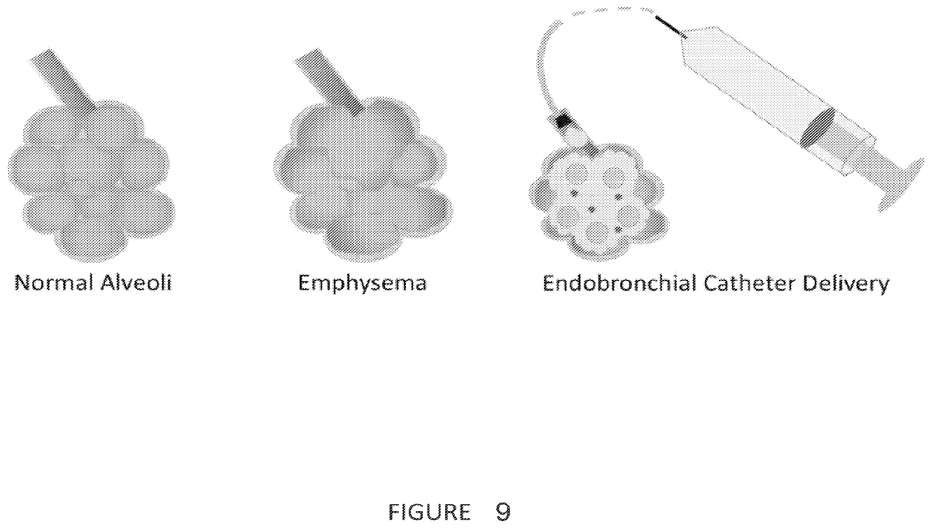

To analyze the integration of human cells into the host rat tissue, we quantified human-specific HLA 1 (h-HLA 1) positive cells and found that h-HLA 1-positive cells represented 14.69%±0.95% of total cells counted per lung (FIG. 2, Panels A through D). h-HLA 1 positive cells distributed across multiple alveoli (FIG. 2, Panel B); on closer look at the alveolar level, we found that single alveoli contained h-HLA 1 positive and h-HLA 1-negative cells of both endothelial (positive: Ulex europaeus agglutinin-I labeled cells-positive) and nonendothelial phenotype (Ulex europaeus agglutinin-I labeled cells-negative) (FIG. 2, Panel C). This was further validated by staining of lungs with anti-GFP for NKX2. 1-GFP cells (FIG. 2, Panels E and F) and human-specific anti-CD3 1 (h-CD3 1) for human endothelial cells (FIG. 2, Panels G and H). In addition, perfusing cell-treated lungs with a tracer (dextran) revealed that iPSC-derived human endothelial cells integrated in perfused vasculature, as determined by h-CD3 1 staining of perfused lung tissue (FIG. 2, Panel C). Furthermore, transmission electron microscope images stained with either h-HLA 1 or h-CD31 suggested the formation of a blood-air barrier by endothelial cells (FIG. 2, Panel J), suggesting the formation of human gas exchange units.

Double staining of both GFP and h-CD31 showed that human transplanted cells may have formed new alveolar units, as determined by the number of GFP- and hCD31-positive cells in randomly selected alveoli (FIG. 2, Panels K and L), both participating in the lining of alveoli (FIG. 2, M). We therefore classified alveoli based on the percentage of human cell incorporation (>70%, >30%, and <30%) in randomly selected regions, and found that human cells in some instances disseminated into the existing host tissue (alveoli with <30% human cells), whereas in other instances contributed to tissue repair (>30% human cells), or may have contributed to new alveolar formation (alveoli with >70% human cells) (FIG. 2, Panel N).

### Effect of Cell Treatment on Progression of Emphysema

To determine whether endothelial cell and pneumocyte injection could reverse or reduce the progression of emphysema, we collected computed tomography scan readings of rat lungs on days 21, 35, and 49. We analyzed the scans for lung volume (−200 to −1200 Hu), vascular density (−300 to −˜50 Hu), and emphysema progression (−800 to −1000 Hu) (FIG. 3, Panel A). Hypodense area representing tissue loss appeared on day 49 postelastase injection, and lung sizes were significantly larger in all disease conditions (cell treated condition=10.27±0.98 mL; P=0.015; emphysema control=10.84±1.25 mL; P=0.009; and vehicle=10.76±1.21 mL; P=0.014) compared with healthy controls (7.25±0.98 mL; 1-way analysis of variance [ANOVA] P=0.006; n=5) (FIG. 3, Panel B). Cell treatment appeared to slow the progress ion of emphysema, as detected by the percentage of the hypodense areas compared with the total lung size (FIG. 3, Panel C), which was 1.54%±0.22% on day 49 in the cell-treated condition versus 3.80%±0.67% (P=0.0002) and 3.46%±0.533% (P˜0.003) in emphysema and vehicle controls, respectively (1-way ANOVA P=0.0007; n=5 each). Vascular density was 47.4%±0.5% of total lung volume in healthy lungs, whereas 32.8%±1.3% in cell-treated lungs (P=0.0001) and 25.6%±1.5% in emphysema control (P=0.0001) and 25.05%±2.4% in vehicle-injected lungs (P=0.0008) (1-way ANOVA P=0.000009). This observation may indicate decreased vascular density in all conditions compared with healthy controls. However, vascular density was significantly higher in cell-treated lungs versus emphysema and vehicle controls (P=0.013 and P=0.020, respectively; n=5 each) (FIG. 3, Panel D), which may suggest increased vascularization after cell treatment. Because this could result from cellular regeneration and proliferation, we examined cellular proliferation in cell-treated lungs by 5-ethynyl-2′-deoxyuridine (EdU) injection. We found that h-HLA1-positive cells had a higher proliferative cell percentage than h-HLA1-negative cells in the same lung, as determined by colocalization of h-HLA1 and EdU, which showed 66.1%±12% of total h-HLA1 positive cells per field. In comparison, rat cells (h-HLA1 negative but EdU-positive) accounted for only 8.1%±2% of total rat cells (P=0.015; n=3) (FIG. 3, Panel E). We further found that the percentage of proliferating h-HLA1 negative cells (host cells) was higher in cell treated animals compared with emphysema controls (8.1%±2% in cell-treated animals vs 5.9%±1.2% in emphysema controls P=0.023; n=5) (FIG. 3, Panel F). Moreover, when analyzing the average alveolar area across conditions via morphometry (FIG. 3, Panel G), we found that it was 324±45 μm² in healthy controls, 453±84 μm² (P=0.17) in cell-treated lungs, 1487±423 μm² in emphysema controls (P=0.0212), and 1315±414 μm² in vehicle controls (P=0.046; 1-way ANOVA P=0.009), suggesting that cell-treatment ameliorated alveolar deterioration. These findings suggest that tissue repair may have been mediated by both rat and human cells. Human cells (h-HLA1-positive) and rat cells (h-HLA1-negative) in cell-treated animals proliferated at a higher rate compared with controls. The observed decrease in emphysema progression and increased vascular density may have been caused by transplanted cells directly through incorporation and proliferation or indirectly by inducing host tissue proliferation in response to injury.

### Cell Treatment Improved Ventilation Mechanics

To determine whether intratracheal injection of endothelial cells and pneumocytes improved functional and ventilation mechanics, we performed isolated lung Cdyn measurements on day 49. On gross examination, all diseased lungs showed evidence of focal bleb formation, confirming emphysema and showing that cell treatment could not reverse the structural breakdown of lung tissue at a macro-scale level (FIG. 4, Panel A). To measure Cdyn, we collected peak PIP readings at 2 mL tidal volume and PEEP of 3 cmH₂O (FIG. 4, Panel B). Cell-treated lungs showed similar compliance as healthy control lungs (0.126±0.002 mL/cmH₂O vs 0.127±0.004 mL/cm H₂O; n=5), respectively; P=0.79; whereas untreated emphysema control lungs and vehicle-treated lungs showed increased Cdyn (0.214±0.002 mL/cmH₂O; P=4.3e-07 and 0.178±0.004 mL/cm H₂O ; P=1.01e-04, respectively, I-way ANOVA P=5.07e-14; n=5 compared with healthy control lungs. Moreover, the residual volume (FIG. 4, Panel C) was smaller in healthy controls and cell-treated lungs (1.61±0.61 mL and 1.87±0.25 mL, respectively) compared with untreated emphysema controls (2.99±0.61 mL) and vehicle-treated lungs (2.92±0.44 mL), respectively (1-way ANOVA P=0.042; cell-treated vs healthy condition P=0.89; cell-treated vs emphysema condition P=0.011; and cell-treated vs vehicle condition P=0.021; n=5). On histologic analysis, we observed elastin deposition in areas of cell engraftment, suggesting that corrected the Cdyn and residual volume could be partly resulting from cell-mediated extracellular matrix repair (FIG. 4, Panel D). To determine whether corrected mechanics were coupled with improved gas exchange, we analyzed arterial blood gas after ventilation with 100% oxygen and room air (FIG. 4, Panel E). We did not observe significant difference between conditions after 100% oxygen exposure: PCO₂) was similar between healthy controls (53.2±5.56 mm Hg), cell treated lungs (48.55±1.79 mm Hg), emphysema controls (51.05±2.01 mm Hg), and vehicle controls (47.42±3.17 mm Hg) (1-way ANOVA P=0.69); P_{O2} was similar between healthy controls (590.3±24.06 mm Hg), cell treated (622.5±19.97 mm Hg), emphysema controls (552.2±26.02 mm Hg), vehicle controls (573.6±30.02 mm Hg) (1-way ANOVA P=0.28). On room air ventilation, P_{CO2} was similar across all groups (healthy controls [35±2.25 mm Hg]), cell treated [39.78±1.09 mm Hg], emphysema controls [39.92±2.07 mm Hg], vehicle controls [41±2.7.4 mm Hg]; 1-way ANOVA P=0.33). However, healthy (117±8.08 mm Hg) and cell-treated lungs (12 1±8.13 mm Hg) achieved a higher P_{O2} compared with emphysema controls (90±4.63 mm Hg), and vehicle-controls (98.5±5.09 mm Hg) (1-way ANOVA P=0.029).

### Discussion

Loss of alveolar units and reduced gas exchange capacity are the hallmarks of pulmonary emphysema that ultimately lead to respiratory failure. To date, no treatment exists to repair or regenerate lost alveolar units in established emphysema. Cells as potential novel therapeutic tools have been proposed to mediate lung repair; however, to date have failed to show effects beyond immunomodulation [Ref. 17, 18].

Due to the recent advances in directed differentiation of distal lung cells from iPSCs, new cell candidates that could rebuild alveolar units have emerged [Ref. 19-23]. Several studies have shown the potential of primary pneumocytes, and alveolar progenitor cells either as single-cell isolate [Ref. 24-26] or organoids [Ref. 27] transplanted either intratracheally [Ref. 24,27] or intravenously, [Ref. 25] to engraft in the host lung, and to differentiate into functional cell phenotypes. Human iPSCs provide an unlimited source of many cellular phenotypes that can acquire tissue-specific functional phenotypes when exposed to tissue formation cues [Ref. 13,28,29]. More recently, detailed observations on the progeny and heterogeneity of cells forming and repairing the alveolar gas exchange unit have emerged [Ref. 30]. Aerocytes (a-Caps) are large endothelial cells responsible for oxygen and carbon dioxide exchange, whereas general capillary cells act as proliferative cells that keep capillary conduits in the alveoli intact upon injury and serve as progenitor cells to a-Cap cells [Ref. 5]. On the luminal side, type 1 pneumocytes line the inner surface of the alveoli and maintain close contact with a-Cap for gas exchange [Ref. 31]. These cells arise from type 2 pneumocytes, which are also responsible for the secretion of lung surfactant [Ref. 12,21,31]. The synergy and necessary crosstalk between capillary endothelial cells and pneumocytes in delivering functional alveoli are also revealed by the interdependent regeneration of a-Cap and type I pneumocytes cells [Ref. 32] after the proliferation and transdifferentiation from capillary endothelia [Ref. 15] and type 2 pneumocytes cells following an injury [Ref. 33]. Taken together, this evidence suggests the need for at least endothelial cells and pneumocytes to regenerate lost alveolar units. In the present Example, we therefore sought to utilize the tremendous progress in iPSC differentiation of pneumocytes and endothelial cells to replace and regenerate lost alveolar units in a rat model of pulmonary emphysema. See FIG. 6 which shows a summary of the Example 1 study of intratracheal transplantation of induced pluripotent stem cells-derived endothelial cells and pneumocytes in an elastase induced emphysema model mediated lung repair by decreasing emphysema progression and improving the vascular density of treated lungs. We found that intratracheal transplantation of iPSC-derived endothelial cells and pneumocytes mediated lung repair by ameliorating emphysema progression and improving the vascular density of treated lungs. We found that human cells disseminated into the host tissue, incorporated in existing alveoli, and helped to regenerate new predominantly human vascularized alveoli as determined by the percentage of human cells and the distribution of both h-CD31 and NKX2.1-GFP markers per individual alveoli. Human cells formed a blood-air barrier as determined by transmission electron microscopy, which confirmed their potential to participate in the formation of structurally intact gas exchange tissue [Ref. 34]. In our dataset, cell treatment ameliorated emphysema progression, which coincided with engraftment and proliferation of transplanted cells and increased proliferation of host cells as determined by EdU incorporation. This finding was paired with decreased alveolar breakdown and improved vascular density after cell treatment possibly indicating structural alveolar repair mediated by transplanted cells. Cell-treated lungs showed improved in vitro compliance compared with untreated controls, which may have been mediated by increased production of extracellular matrix proteins such as elastin and collagen. Both provide the lung with elasticity and tensile strength and are usually only expressed during lung development and growth [Ref. 35]. On room air ventilation, cell treatment improved oxygenation, but did not correct the observed mild hypercapnia, which could be related to ongoing inflammation and reactive hypoventilation after elastase treatment [Ref. 36]. In summary, our Example 1 shows the feasibility and safety of intratracheal iPSC-derived endothelial and pneumocyte delivery in a rat emphysema model. We found evidence that cells could ameliorate emphysema structurally (by integrating into the host tissue and forming blood vessels) and functionally (by improving emphysema progression and ventilation). Further work is envisioned to better understand the long-term fate of transplanted cells and to optimize cell number and treatment frequency toward clinical use.

### Example 1 References

1. Melo-Narváez M. C., et al., Lung regeneration: implications of the diseased niche and ageing. Eur Respir Rev. 2020; 29:1-13.
2. Houghton A. M. G., et al., Elastin fragments drive disease progression in a murine model of emphysema. J Clin Invest. 2006; 116:753-759.
3. Müller K. C., et al., Lung fibroblasts from patients with emphysema show markers of senescence in vitro. Respir Res. 2006; 7:1-10.
4. Janoff A., et al., Elastases and emphysema. Current assessment of the protease-antiprotease hypothesis. Am Rev Respir Dis. 1985; 132:417-433.
5. Gillich A., et al., Capillary cell-type specialization in the alveolus. Nature. 2020; 586:785-789.
6. Snider G. L., et al., The definition of emphysema Report of a National Heart, Lung, and Blood Institute, Division of Lung Diseases Workshop. Am Rev Respir Dis. 1965; 132:182-185.
7. Cunningham D., et al., A Randomized trial comparing lung-volume-reduction surgery with medical therapy for severe emphysema. N Engl J Med. 2003; 348:2059-2073.
8. Ng-Blichfeldt J. P., et al., Regenerative pharmacology for COPD: breathing new life into old lungs. Thorax.2019; 74:890-897.
9. Oh D. K., et al., Lung regeneration therapy for chronic obstructive pulmonary disease. Tuberc Respir Dis. 2017; 80:1-10.
10. Becerra D., et al., High-throughput culture method of induced pluripotent stem cell-derived alveolar epithelial cells. Tissue Eng C Methods. 2021; 27:639-648.
11. Jacob A., et al., Differentiation of human pluripotent stem cells into functional lung alveolar epithelial cells. Cell Stem Cell. 2017; 21:472-488.
12. Jacob A., et al., Derivation of self-renewing lung alveolar epithelial type II cells from human pluripotent stem cells. Nat Protoc. 2019; 14:3303-3332.
13. Ren X., et al., Engineering pulmonary vasculature in decellularized rat and human lungs. Nat Biotechnol. 2015; 33:1097-1102.
14. Wimmer R. A., et al., Generation of blood vessel organoids from human pluripotent stem cells. Nat Protoc. 2019; 14:3082-3100.
15. Ott H. C., et al., Regeneration and orthotopic transplantation of a bioartificial lung. Nat Med. 2010; 16:927-933.
16. Becerra D., et al., Characterization of an elastase-induced emphysema model in immune-deficient rats. Eur J Cardio Thorac Surg.2021; 59:309-315.
17. Sipp D., et al., Clear up this stem-cell mess. Nature. 2018; 561:455-457.
18. Kotton D., et al., Failure of bone marrow to reconstitute lung epithelium. Am J Respir Cell Mol Biol. 2005; 33:328-334.
19. Basil M. C., et al., Human distal airways contain a multipotent secretory cell that can regenerate alveoli. Nature. 2022; 604:120-126.
20. Cao Z., et al., Targeting of the pulmonary capillary vascular niche promotes lung alveolar repair and ameliorates fibrosis. Nat Med. 2016; 22:154-162.
21. Basil M. C., et al., The cellular and physiological basis for lung repair and regeneration: past, present, and future. Cell Stem Cell. 2020; 26:482-502.
22. Zepp J. A., et al., Genomic, epigenomic, and biophysical cues controlling the emergence of the lung alveolus. Science. 2021; 371: eabc3172.
23. Alysandratos K. D., et al., Epithelial stem and progenitor cells in lung repair and regeneration. Annu Rev Physiol. 2021; 83:529-550.
24. Kathiriya J. J., et al., Distinct airway epithelial stem cells hide among club cells but mobilize to promote alveolar regeneration. Cell Stem Cell. 2020; 26:346-358.e4.
25. Hillel-Karniel C., et al., Multi-lineage lung regeneration by stem cell transplantation across major genetic barriers. Cell Rep. 2020; 30:807-819.e4.
26. Milman Krentsis I., et al., Lung injury repair by transplantation of adult lung cells following preconditioning of recipient mice. Stem Cells Transl Med. 2018; 7:68-77.
27. Louie S. M., et al., Progenitor potential of lung epithelial organoid cells in a transplantation model. Cell Rep. 2022; 39:110662.
28. Wimmer R. A., et al., Human blood vessel organoids as a model of diabetic vasculopathy. Nature. 2019; 565:505-510.
29. Homan K. A., et al., Flow-enhanced vascularization and maturation of kidney organoids in vitro. Nat Methods. 2019; 16:255-262.
30. Sun X., et al., A census of the lung: CellCards from LungMAP. Dev Cell. 2022; 57:112-145.e2.
31. Hsia C. C. W., et al., Lung structure and gas exchange. Physiol Behav. 2016; 176:100-106.
32. Ellis L. V., et al., Epithelial VEGFA specifies a distinct endothelial population in the mouse lung Lisandra. Physiol Behav. 2016; 176:139-148.
33. Zacharias W. J., et al., Regeneration of the lung alveolus by an evolutionarily conserved epithelial progenitor. Nature. 2018; 555:251-255.
34. Weibel E. R., et al., Morphological basis of alveolar-capillary gas exchange. Physiol Rev. 1973; 53:419-495.
35. Toshima M, et al., Three-dimensional architecture of elastin and collagen fiber networks in the human and rat lung. Arch Histol Cytol. 2004; 67:31-40.
36. Richter D., et al., Mechanisms of respiratory rhythm generation. Curr Opin Neurobiol. 1992; 2:788-793.
   The citation of any document or reference is not to be construed as an admission that it is prior art with respect to the present invention.

### Example 2

### Overview of Example 2

Here, a novel injectable hydrogel/microsphere platform is described to serve as a scaffold for lung tissue regeneration in patients with emphysema by providing a biocompatible, biodegradable alveolus-like structure. The bulk hydrogel platform utilized here comprises gelatin fibers modified to include the click moieties such as tetrazine (T) and norbornene (N) for quick gelation upon mixing and delivery in physiologic environments as previously described [Ref. 1]. Within this bulk hydrogel, it is uniquely proposed to incorporate alginate microspheres in the range of 150 to 250 μm. Alginate microparticles will be incorporated within the bulk gel to provide alveolar-like structures to aid in cell organization. For controlled degradation of alginate microspheres, alginate lyase will be incorporated within the bulk gel.

To develop a clinically translatable scaffold, two innovations were necessary: (1) to incorporate a bulk gel that is injectable, and (2) to achieve controlled degradation of the alginate microspheres. These objectives can be achieved through the use of a click hydrogel which gels upon mixing and by the incorporation of an alginate-specific enzyme for controlled degradation of the incorporated microspheres. The composite material scaffold as well as the mode of application are novel in the art.

### Introduction to Example 2

Pulmonary emphysema is characterized by the permanent enlargement of distal airspaces causing destruction of the fragile tissue in the air sacs and air trapping within the lungs. While there are treatment options to manage the disease, there does not exist a cure. Existing procedures to treat the disease such as lung transplantation and lung volume reduction surgery present high risk, and there is a need for minimally invasive and more effective treatment options. Here, a novel injectable hydrogel/microsphere platform is described to serve as a scaffold for lung tissue regeneration in patients with emphysema by providing a biocompatible, biodegradable alveolus-like structure.

The bulk hydrogel platform utilized here comprises gelatin fibers modified to include the click moieties tetrazine (T) and norbornene (N) for quick gelation upon mixing and delivery in physiologic environments (see FIG. 7). Within this bulk hydrogel, it is uniquely proposed to incorporate alginate microspheres in the range of 150 to 250 μm. (see FIG. 8). Alginate is a well-characterized and biocompatible biomaterial. Due to its mild gelation conditions, structural stability, and immunoprotective barrier alginate gels have been implemented for cell entrapment [Ref. 2]. Most notably, alginate has been used in transplantation of pancreatic islet cells to treat Type I diabetes. However, alginate, which is derived from brown algae, does not possess inherent cell adhesive ligands nor does it exhibit biodegradation in vivo.

Previous attempts to create alveolus-like constructs in hydrogels include a photodegradable microsphere template [Ref. 3] as well as a collagen: Matrigel sheet containing alginate microparticles as space-filling cyst-like structures [Ref. 4]. While the engineered photodegradable cyst structures were sufficient for in vitro formation of alveolar spheres, photocleavage is not suitable for in vivo application due to limitations in light penetration. The space-filling method proposed by [Ref. 4] resulted in persistence of alginate particles and significant cell death after 21 days. Additionally, the collagen: Matrigel bulk gel which they described is not suited to gelation at injection. To develop a clinically translatable scaffold, two innovations are necessary: (1) to incorporate a bulk gel that is injectable, and (2) to achieve controlled degradation of the alginate microspheres. Therefore, a click gelatin hydrogel is proposed for optimal delivery, cell infiltration and migration, as well as biodegradation. Alginate microparticles will be incorporated within the bulk gel to provide alveolar-like structures to aid in cell organization. For controlled degradation of alginate microspheres, alginate lyase will be incorporated within the bulk gel. (See FIG. 9.)

### Example 2 References

1. Koshy et.al, “Click-Crosslinked Injectable Gelatin Hydrogels”, Adv. Healthc. Mater., 2016 March 9;5(5):541-7.
2. Szekalska et.al, “Alginate: Current Use and Future Perspectives in Pharmaceutical and Biomedical Applications”, International Journal of Polymer Science, vol. 2016, Article ID 7697031, 2016.
3. Lewis et.al, “In vitro model alveoli from photodegradable microsphere templates”, Biomater. Sci.,2015, 3, 821-832.
4. Zhang et.al., “The reconstruction of lung alveolus-like structure in collagen-matrigel/microcapsules scaffolds in vitro,” J Cell Mol Med. 2011 September;15(9):1878-1886.
   The citation of any document or reference is not to be construed as an admission that it is prior art with respect to the present invention.

Thus, the present invention provides methods and compositions for tissue regeneration, and more particularly to methods and compositions for lung tissue regeneration. The present disclosure also provides hydrogel-microsphere scaffolds for generating specific tissue structures. More specifically, the disclosure also provides click chemistry-driven hydrogel formation with degradable microspheres as scaffolds for generating alveolus-like structures.

In light of the principles and example embodiments described and illustrated herein, it will be recognized that the example embodiments can be modified in arrangement and detail without departing from such principles. Also, the foregoing discussion has focused on particular embodiments, but other configurations are also contemplated. In particular, even though expressions such as “in one embodiment”, “in another embodiment”, “in other embodiments”, “in some embodiments”, or the like are used herein, these phrases are meant to generally reference embodiment possibilities, and are not intended to limit the invention to particular embodiment configurations. As used herein, these terms may reference the same or different embodiments that are combinable into other embodiments. As a rule, any embodiment referenced herein is freely combinable with any one or more of the other embodiments referenced herein, and any number of features of different embodiments are combinable with one another.

Although the invention has been described in considerable detail with reference to certain embodiments, one skilled in the art will appreciate that the present invention can be used in alternative embodiments to those described, which have been presented for purposes of illustration and not of limitation. Therefore, the scope of the appended claims should not be limited to the description of the embodiments contained herein.

What is claimed is:

1. A method for regenerating and/or repairing lung tissue in a subject, the method comprising:
administering to a subject in need of lung tissue regeneration and/or repair a composition including (i) a carrier comprising a scaffold-forming material, (ii) cellular material selected from the group consisting of endothelial cells, epithelial cells, mesenchymal stem cells, and mixtures thereof, and (iii) pneumocytes.

2. The method of claim 1 wherein:
the cellular material comprises endothelial cells.

3. The method of claim 2 wherein:
the endothelial cells comprise induced pluripotent stem cell-derived endothelial cells.

4. The method of claim 2 wherein:
the endothelial cells are cultured prior to administration of the composition.

5. The method of claim 1 wherein:
the pneumocytes comprise induced pluripotent stem cell-derived pneumocytes.

6. The method of claim 1 wherein:
the pneumocytes comprise surfactant protein-C-positive pneumocytes.

7. The method of claim 1 wherein:
the scaffold-forming material comprises a hydrogel.

8. The method of claim 1 wherein the scaffold-forming material comprises:
a biopolymer having a first reactive group and a second reactive group, wherein the first reactive group and the second reactive group react via click chemistry to crosslink the biopolymer to form a hydrogel;
a porogen; and
a porogen-degrading agent.

9. The method of claim 8 wherein:
the biopolymer comprises gelatin,
the porogen comprises polysaccharide microspheres, and
the porogen-degrading agent comprises a polysaccharide-lyase.

10. The method of claim 9 wherein:
the polysaccharide microspheres comprise alginate microspheres, and
the polysaccharide-lyase comprises alginate-lyase.

11. The method of claim 1 wherein the scaffold-forming material comprises:
a first biopolymer having a first reactive group;
a second biopolymer having a second reactive group, wherein the first reactive group and the second reactive group react via click chemistry to crosslink the first biopolymer and the second biopolymer to form a hydrogel;
a porogen; and
a porogen-degrading agent.

12. The method of claim 11 wherein:
one or both of the first biopolymer and the second biopolymer comprises gelatin,
the porogen comprises polysaccharide microspheres, and
the porogen-degrading agent comprises a polysaccharide-lyase.

13. The method of claim 12 wherein:
the polysaccharide microspheres comprise alginate microspheres, and
the polysaccharide-lyase comprises alginate-lyase.

14. The method of claim 1 wherein:
the administering is intravenously or intratracheally.

15. The method of claim 1 wherein:
the administering is via airways to the lung.

16. The method of claim 1 wherein:
the lung tissue is emphysematous, and
following the administration of the composition, emphysema progression is ameliorated and vascular density of lungs is improved.

17. The method of claim 1 wherein:
the lung tissue is emphysematous, and
the method ameliorates emphysema structurally by integrating into host lung tissue and forming blood vessels, and functionally by improving emphysema progression and ventilation.

18. The method of claim 1 wherein:
the lung tissue is emphysematous, and
following the administration of the composition, transplanted cells engrafted in at least 10% of host alveoli and fully integrated to form vascularized alveoli together with host cells.

19. An injectable composition for forming a scaffold, the composition comprising:
a carrier comprising a scaffold-forming material;
cellular material selected from the group consisting of endothelial cells, epithelial cells, mesenchymal stem cells, and mixtures thereof; and
pneumocytes.

20. The composition of claim 19 wherein:
the cellular material comprises endothelial cells.

21. The composition of claim 20 wherein:
the endothelial cells comprise induced pluripotent stem cell-derived endothelial cells.

22. The composition of claim 20 wherein:
the endothelial cells are cultured prior to administration of the composition.

23. The composition of claim 19 wherein:
the pneumocytes comprise induced pluripotent stem cell-derived pneumocytes.

24. The composition of claim 19 wherein:
the pneumocytes comprise surfactant protein-C-positive pneumocytes.

25. The composition of claim 19 wherein:
the scaffold-forming material comprises a hydrogel.

26. The composition of claim 19 wherein the scaffold-forming material comprises:
a biopolymer having a first reactive group and a second reactive group, wherein the first reactive group and the second reactive group react via click chemistry to crosslink the biopolymer to form a hydrogel;
a porogen; and
a porogen-degrading agent.

27. The composition of claim 26 wherein:
the biopolymer comprises gelatin,
the porogen comprises polysaccharide microspheres, and
the porogen-degrading agent comprises a polysaccharide-lyase.

28. The composition of claim 27 wherein:
the polysaccharide microspheres comprise alginate microspheres, and
the polysaccharide-lyase comprises alginate-lyase.

29. The composition of claim 19 wherein the scaffold-forming material comprises:
a first biopolymer having a first reactive group;
a second biopolymer having a second reactive group, wherein the first reactive group and the second reactive group react via click chemistry to crosslink the first biopolymer and the second biopolymer to form a hydrogel;
a porogen; and
a porogen-degrading agent.

30. The composition of claim 29 wherein:
one or both of the first biopolymer and the second biopolymer comprises gelatin,
the porogen comprises polysaccharide microspheres, and
the porogen-degrading agent comprises a polysaccharide-lyase.

31. The composition of claim 30 wherein:
the polysaccharide microspheres comprise alginate microspheres, and
the polysaccharide-lyase comprises alginate-lyase.

32. A kit for use in in producing a tissue scaffold, the kit comprising:
a container; and
an amount of the composition of any of claims 19-31 in the container.

33. The kit of claim 32 wherein:
the container is a medical syringe.

34. A kit for use in in producing a tissue scaffold, the kit comprising:
a container; and
an amount of the composition of claim 19 in the container, wherein the cellular material comprises endothelial cells,
wherein the endothelial cells are present in the amount of the composition of claim 19 in a range of 10 million to 500 million endothelial cells based on a total amount of the composition of claim 19 in the container, and
wherein the pneumocytes are present in the amount of the composition of claim 19 in a range of 1 million to 500 million pneumocytes based on the total amount of the composition of claim 19 in the container.

35. A composition according to any of claims 19-31 for use in the treatment of a lung condition.

36. A composition according to any of claims 19-31 for use in the treatment of emphysema.

37. An injectable composition for forming a scaffold, the composition comprising:
a biopolymer having a first reactive group and a second reactive group, wherein the first reactive group and the second reactive group react via click chemistry to crosslink the biopolymer to form a hydrogel;
polysaccharide microspheres; and
a polysaccharide-lyase.

38. The composition of claim 37 wherein:
the biopolymer comprises gelatin.

39. The composition of claim 37 wherein:
the biopolymer comprises gelatin fibers.

40. The composition of claim 37 wherein:
the polysaccharide microspheres comprise alginate microspheres, and
the polysaccharide-lyase comprises alginate-lyase.

41. The composition of claim 37 wherein:
the microspheres have a maximal dimension in a range of 150 μm to 250 μm.

42. The composition of claim 37 further comprising:
cellular material selected from the group consisting of endothelial cells, epithelial cells, mesenchymal stem cells, and mixtures thereof; and
pneumocytes.

43. The composition of claim 42, wherein the cellular material comprises endothelial cells.

44. The composition of claim 37 wherein:
the first reactive group is tetrazine and the second reactive group is norbornene.

45. A kit for use in in producing a tissue scaffold, the kit comprising:
a container; and
an amount of the composition of any of claims 37-44 in the container.

46. The kit of claim 45 wherein:
the container is a medical syringe.

47. A kit for use in in producing a tissue scaffold, the kit comprising:
a container; and
an amount of the composition of claim 43 in the container,
wherein the endothelial cells are present in the amount of the composition of claim 43 in a range of 10 million to 500 million endothelial cells based on a total amount of the composition of claim 43 in the container, and
wherein the pneumocytes are present in the amount of the composition of claim 43 in a range of 1 million to 500 million pneumocytes based on the total amount of the composition of claim 43 in the container.

48. A composition according to any of claims 37-44 for use in the treatment of a lung condition.

49. A composition according to any of claims 37-44 for use in the treatment of emphysema.

50. An injectable composition for forming a scaffold, the composition comprising:
a first biopolymer having a first reactive group;
a second biopolymer having a second reactive group, wherein the first reactive group and the second reactive group react via click chemistry to crosslink the first biopolymer and the second biopolymer to form a hydrogel;
polysaccharide microspheres; and
a polysaccharide-lyase.

51. The composition of claim 50 wherein:
one or both of the first biopolymer and the second biopolymer comprises gelatin.

52. The composition of claim 50 wherein:
both the first biopolymer and the second biopolymer comprise gelatin.

53. The composition of claim 50 wherein:
one or both of the first biopolymer and the second biopolymer comprises gelatin fibers.

54. The composition of claim 50 wherein:
the polysaccharide microspheres comprise alginate microspheres, and
the polysaccharide-lyase comprises alginate-lyase.

55. The composition of claim 50 wherein:
the microspheres have a maximal dimension in a range of 150 μm to 250 μm.

56. The composition of claim 50 further comprising:
cellular material selected from the group consisting of endothelial cells, epithelial cells, mesenchymal stem cells, and mixtures thereof; and
pneumocytes.

57. The composition of claim 56, wherein the cellular material comprises endothelial cells.

58. The composition of claim 50 wherein:
the first reactive group is tetrazine and the second reactive group is norbornene.

59. A kit for use in in producing a tissue scaffold, the kit comprising:
a container; and
an amount of the composition of any of claims 50-58 in the container.

60. The kit of claim 59 wherein:
the container is a medical syringe.

61. A kit for use in in producing a tissue scaffold, the kit comprising:
a container; and
an amount of the composition of claim 57 in the container,
wherein the endothelial cells are present in the amount of the composition of claim 57 in a range of 10 million to 500 million endothelial cells based on a total amount of the composition of claim 57 in the container, and
wherein the pneumocytes are present in the amount of the composition of claim 57 in a range of 1 million to 500 million pneumocytes based on the total amount of the composition of claim 57 in the container.

62. A composition according to any of claims 50-58 for use in the treatment of a lung condition.

63. A composition according to any of claims 50-58 for use in the treatment of emphysema.

64. A therapeutic method of providing a scaffold in a tissue environment in the body of a subject, the method comprising:
injecting the composition of any of claims 19-31 or any of claims 37-44 or any of claims 50-58 into the tissue environment; and
allowing the composition to solidify to a scaffold and degrade.

65. The method of claim 64 wherein:
the scaffold comprises a biodegradable, biocompatible alveolus-like structure.

66. The method of claim 64 wherein:
the tissue comprises lung tissue.

67. The method of claim 64 wherein:
the injecting is intratracheally into the lung(s) of the subject.

68. The method of claim 64 wherein the method is a therapeutic treatment for emphysema.
